(19) United States
(12) Patent Application Publication
(10) Pub. No.: US 2024/0050746 A1
(43) Pub. Date: Feb. 15, 2024

(54) SYSTEMS AND METHODS FOR IMPROVING MULTIPLE PHYSIOLOGICAL FUNCTIONS USING NEUROSTIMULATION

(71) Applicant: UNIVERSITY OF LOUISVILLE RESEARCH FOUNDATION, INC., Louisville, KY (US)

(72) Inventors: Claudia Angeli, Louisville, KY (US); Charles Hubscher, Louisville, KY (US); Gail F. Forrest, Cranbury, NJ (US); Enrico Rejc, Louisville, KY (US); Susan J. Harkema, Louisville, KY (US); Maxwell Boakye, Louisville, KY (US); Yury Gerasimenko, Louisville, KY (US)

(21) Appl. No.: 18/447,661

(22) Filed: Aug. 10, 2023

Related U.S. Application Data
(60) Provisional application No. 63/371,128, filed on Aug. 11, 2022.

(51) Int. Cl. A61N 1/36; A61B 5/388

(52) U.S. Cl. CPC A61N 1/36062; A61B 5/388; A61N 1/36139

(57) ABSTRACT

The present invention relates to systems and methods for improvement of physiological function in an individual with a spinal cord injury via electrical stimulation of the spinal cord. A 3D model of the spinal cord is used to determine an initial position for an electrode array to deliver stimulation. Spinal cord electrical stimulation is configurable to target different physiological functions with surgical implantation of the neurostimulator at a singular location. Such systems and methods include (i) anatomical specificity to target the appropriate region of the spinal cord for stimulation, (ii) electrical specificity to provide the appropriate stimulation by delivery method, frequency, pulse duration, and other factors, and (iii) physiological specificity to evoke, suppress, increase, or decrease a specific physiological result.

### CROSS-REFERENCE TO RELATED APPLICATION

This application claims the benefit of priority to U.S. provisional patent application Ser. No. 63/371,128, filed Aug. 11, 2022, for SYSTEMS AND METHODS FOR IMPROVING MULTIPLE PHYSIOLOGICAL FUNCTIONS USING NEUROSTIMULATION, incorporated herein by reference.

### GOVERNMENT SUPPORT

This invention was made with government support under grant numbers 1R01NS102920-01A1, 1R01HL150581-01A1, 2R01 HD080205-06, 3OT2OD024898-01S5, 1UH3NS116238-01, 1R01EB007615, and 1OT2OD024898 each of which were awarded by the National Institutes of Health. The government has certain rights in the invention.

### FIELD OF THE INVENTION

The present invention relates to systems and methods for improvement of physiological function in an individual with a spinal cord injury via electrical stimulation of the spinal cord. Spinal cord electrical stimulation is configurable to target different physiological functions, including one or more of lower extremity voluntary motor movement, upper extremity voluntary motor movement, posture, locomotion, bladder function, bowel function, cardiovascular function, and respiratory function, with surgical implantation of the neurostimulator at a singular location or transcutaneous stimulation at multiple locations. Such systems and methods include (i) anatomical specificity to target the appropriate region of the spinal cord for stimulation, (ii) electrical specificity to provide the appropriate stimulation by delivery method, frequency, pulse duration, and other factors, and (iii) physiological specificity to evoke, suppress, increase, or decrease a specific physiological result.

### BACKGROUND OF THE INVENTION

Spinal cord injury (SCI) often leads to damage of ascending and descending spinal tracts, interrupting the flow of information to and from the brain. This can result in partial paralysis if some tracts are preserved and complete paralysis if none or a small percentage are preserved. SCI can negatively affect multiple physiological functions, depending on the location and severity of the injury, including lower extremity voluntary motor movement, upper extremity voluntary motor movement, posture, locomotion, bladder function, bowel function, cardiovascular function, and respiratory function. Research in the field, as well as diagnostic and therapeutic methods, typically focus on a single physiological function. Accordingly, a global approach to improving physiological function in individuals with SCI would be both highly desirable and beneficial.

Emerging clinical data indicate that neuromodulation of the lumbosacral spinal cord restores motor and autonomic functions in those with chronic SCI. These discoveries provide evidence for the realistic potential of epidural stimulation to significantly improve function, health, and quality of life of those with chronic clinically incomplete and even complete SCI. However, a lack of clear guidelines on stimulation parameters is perceived as a significant barrier toward the use of spinal cord epidural stimulation for functional improvement.

Two approaches to epidural neuromodulation of the lumbosacral spinal cord have shown motor and autonomic recovery for those with chronic spinal cord injury. One approach, spinal cord epidural stimulation (scES) involves single placement of a 16-electrode array below the level of injury over the lumbosacral enlargement, a widened area of the spinal cord that give attachment to the nerves which supply the lower limbs. An attached pulse generator positioned dorsally (subcutaneously) utilizes tonic-interleaved configurations (anode and cathode contact selection; stimulation frequency, pulse width, and amplitude) that targets optimization of the excitability state of the lumbosacral spinal cord enabling the integration of appropriate sensory information and residual supraspinal influences to restore motor and autonomic function with integrated regulatory control. Another approach, epidural electrical stimulation (EES), uses patterned spatial and temporal stimulation of the dorsal roots of the lumbosacral spinal cord specific for motor function and a separate placement in the thoracic spinal cord for blood pressure regulation.

To date, the major challenges related to the selection of stimulation parameters for promoting motor and autonomic function recovery in SCI individuals are that i) these parameters are task- and individual-specific; and ii) the combination of stimulation parameters potentially available is vast, as exemplified by the over 43 million different electrode configurations available when using a 16-electrode array.

### SUMMARY

The instant subject matter relates to systems and methods for improvement of physiological function in an individual with a spinal cord injury via electrical stimulation of the spinal cord. Such systems and methods include (i) anatomical specificity to target the appropriate region of the spinal cord for stimulation, (ii) electrical specificity to provide the appropriate stimulation by delivery method, frequency, pulse duration, and other factors, and (iii) physiological specificity to evoke, suppress, increase, or decrease a specific desired physiological result. In some embodiments, multiple regions of the spinal cord may be subject to electrical stimulation for concurrent improvement of multiple physiological functions.

In some embodiments, the disclosed invention relates to sequential treatment of multiple physiological functions using different stimulation parameters provided by a stimulation source (implanted neurostimulator, transcutaneous stimulation, or a combination thereof). An individual with spinal cord injury typically has deficiencies in multiple physiological functions, e.g., bowel, bladder, cardiovascular (“CV”), motor, respiratory, and others. Prior techniques focused on providing spinal cord electrical stimulation to improve physiological function of one areas, or at most, two areas, e.g., CV and one other area, such as bladder or motor function, at substantially the same time. However, an individual may need different physiological functions to be addressed sequentially over the course of the day. For example, an individual's day may including standing up (motor+CV), followed by voiding (bladder and/or bowel+CV), followed by eating a meal (CV only), followed by exercise (CV+respiratory+upper extremity), followed by further activity. Spinal cord stimulation is required to facilitate desired functionality of each physiological function. However, regions of the spine which receive stimulation applicable to one physiological function may overlap or not overlap with other regions of the spine applicable to other physiological function, so positioning of the electrical stimulator (scES and/or scTS) preferably accounts for all physiological functions and stimulation modalities contemplated for the individual.

In other embodiments, the disclosed invention relates to configuring functionally focused scES or scTS by the following: 1) Neuroanatomical 3D spinal cord reconstruction model and intra-operative evoked potentials or transcutaneous evoked potentials are used to inform electrode lead placement; 2) spatial-temporal electrophysiological mapping is used to inform motor and autonomic mapping; and 3) task specific motor mapping (leg movements, standing and stepping) and autonomic mapping (blood pressure regulation, bladder capacity and initiation of bladder void). Immediate integrated movement and physiological responses to scES or scTS indicates the sophistication of the human lumbosacral spinal circuitry with the critical role for ongoing regulation of motor and autonomic function.

It will be appreciated that the various systems and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A better understanding of the present invention will be had upon reference to the following description in conjunction with the accompanying drawings.

FIG. 1A depicts an exemplary embodiment of a method for surgical paddle electrode placement including: Panel A showing a preoperative MRI-based Neuroanatomical 3D reconstruction of spinal cord with scES paddle placement; Panel B showing a spinal cord MRI in sagittal view; Panel C showing a high resolution axial MRI scans; and Panel D showing an intraoperative fluoroscopy image with red dashed lines showing integration of X-ray and MRI scans to estimate the paddle placement with respect to the spinal cord levels.

FIG. 1B depicts a first electrode combination of an exemplary intraoperative spatio-temporal mapping shown with overlaid evoked potentials and recruitment curves of muscle responses to the stimulation with various electrode combinations from the scES paddle array. EMG is recorded from rectus femoris (RF), medial hamstrings (MH), tibialis anterior (TA), medial gastrocnemius (MG), soleus (SOL).

FIG. 1C depicts intraoperative spatio-temporal mapping for an exemplary first scES paddle electrode placement at L3-S lumbosacral spinal cord during placement adjustment. Left to right, the figure depicts a first stimulation pattern for the paddle electrode at the first placement, evoked potential curves for the first pattern, recruitment curves for the first pattern, an representative illustration of the first placement on the spinal cord, evoked potential curves for the second pattern, recruitment curves for the second pattern, and a second stimulation pattern for the paddle electrode at the first placement. EMG is recorded from rectus femoris (RF), medial hamstrings (MH), tibialis anterior (TA), medial gastrocnemius (MG), and soleus (SOL).

FIG. 1D depicts an exemplary intraoperative spatio-temporal mapping for a second scES paddle electrode placement at L2-L5 lumbosacral spinal cord during placement adjustment. Left to right, the figure depicts a first stimulation pattern for the paddle electrode at the second placement, evoked potential curves for the first pattern, recruitment curves for the first pattern, an representative illustration of the second placement on the spinal cord, evoked potential curves for the second pattern, recruitment curves for the second pattern, and a second stimulation pattern for the paddle electrode at the second placement. EMG is recorded from rectus femoris (RF), medial hamstrings (MH), tibialis anterior (TA), medial gastrocnemius (MG), and soleus (SOL).

FIG. 2A depicts an exemplary spatio-temporal mapping including colormaps with peak-to-peak amplitude values (pV) corresponding to stimulation current and electrode configuration (numbers in each row refer to contacts of the electrode array tested shown to the left. All combinations are in the middle column of the electrode array. Selected left (top) and right (bottom) muscles: rectus femoris (RF), medial hamstrings (MH), tibialis anterior (TA), soleus (SOL) are shown.

FIG. 2B depicts an exemplary spatio-temporal mapping including colormaps with peak-to-peak amplitude values (pV) corresponding to stimulation current and electrode configuration (numbers in each row refer to contacts of the electrode array tested shown to the left. All combinations are in the lateral columns of the electrode array. Left tibials antereior (LTA) (top) and right tibialis anterior (RTA)(bottom) are shown.

FIG. 2C depicts an exemplary spatio-temporal mapping including EMG responses for left and right muscles for a caudal wide-field frequency response. Vertical lines identify the time of frequency increase while the amplitude remains constant at 6.5 mA.

FIG. 3A depicts a representative flowchart of the steps for selection of scES parameters for voluntary movement. Spatio-temporal mapping is used to identify the areas of the electrode array where tibialis anterior (TA) and iliopsoas (IL) activity is present. An initial configuration is represented as the data (parallelogram), decisions in the process are represented by rhombus shapes. If the response to the question is YES you continue down the flowchart if NO the flowchart will guide you to the restarting point. A predefined process (rectangles) will expand on the assessments needed to optimize the configuration and it is typically followed by a decision. An alternate process (rounded corner rectangles) can be inserted late in the flow chart if a complex decision is not achieved promoting a small modification to the configuration and restarting linking back to a predetermined process. The asterisk (*) signifies a stopping point if full range of motion cannot be achieved during the mapping process.

FIG. 3B depicts an exemplary color map with peak-to-peak amplitude values (pV) corresponding to stimulation current and electrode configuration (numbers in each row refer to contacts of the electrode array) tested for the iliopsoas (IL), vastus lateralis (VL), and tibialis anterior (TA) muscles.

FIG. 3C shows graphs depicting EMG activity of representative leg muscles during 30 Hz spatio-temporal mapping at three different electrode locations. Epidural stimulation programs (P) applied are reported to the right side of each panel. Amplitude is ranges are the same in all three locations producing different activation patterns.

FIG. 3D shows graphs depicting EMG activity of representative leg muscles during amplitude increases and voluntary attempts in a mapping session. Bottom panel represents the optimal configuration at 6.5 mA, generating appropriate range of motion. Epidural stimulation program is reported at the bottom panel and it is the same for all attempts.

FIG. 4A depicts a representative decision-making flowchart for the selection of stimulation parameters for standing. Spatio-temporal mapping assessed in supine position is used to identify the electrode array contacts, and the related polarity, eliciting the activation of knee extensors (KE) and plantar flexors (PF) while limiting the activation of medial hamstrings (MH). After the initial stimulation configuration (parallelogram) is defined, decisions in the process are represented by rhombus shapes. If the response to the question is YES the flow continues vertically down; if NO the flowchart will guide you to the restarting point. A predefined process (rectangle) will expand on the assessment needed to optimize the configuration and it is followed by a decision. If the decision outcome is negative, an alternative process (rounded corner rectangle) is included to allow for small adjustments of the stimulation parameters and restarting back to a predetermined process. In case the mapping process does not result in the targeted functional outcome (achieve independent lower limb extension), stimulation configuration may be determined at the flow-chart level identified with the asterisk (*).

FIG. 4B depicts an exemplary color map with peak-to-peak amplitude values (μV) corresponding to a representative stimulation current and electrode configuration (numbers in each row refer to contacts of the electrode array reported in FIG. 4A) tested for the right (R) medial hamstrings (MH), rectus femoris (RF), vastus lateralis (VL) and soleus (SOL) muscles.

FIG. 4C shows epidural stimulation programs (P) and resulting EMG activity of representative right and left leg muscles during a first stand mapping session.

FIG. 4D shows epidural stimulation programs (P) and resulting EMG activity of representative right and left leg muscles during a second stand mapping session. Independent extension (Ind.) of the left (L) leg is indicated by the time windows within vertical dotted lines.

FIG. 4E shows epidural stimulation programs (P) and resulting EMG activity of representative right and left leg muscles during a third stand mapping session. Independent extension (Ind.) of both legs (BL) concurrently is indicated by the time windows within vertical dotted lines. In this figure, P3 was added to increase the participant's blood pressure because persistent low blood pressure experienced by the participant limited standing bouts duration.

FIG. 4F includes graphs of exemplary EMG variables (IEMG: integrated EMG; MDF: median frequency; MDF SD: median frequency standard deviation) associated with standing ability in this population.

FIG. 5A depicts a representative decision making flowchart for the selection of scES parameters for stepping. Spatio-temporal mapping is used to identify the areas of the electrode array where tibialis anterior (TA) and iliopsoas (IL) activity is present, as well as extensor activity. In addition the lower extremity voluntary mapping and standing mapping are used to examine optimal and suboptimal configuration. An initial configuration is represented as the data (parallelogram), decisions in the process are represented by rhombus shapes. If the response to the question is YES you continue down the flowchart if NO the flowchart will guide you to the restarting point. A predefined process (rectangles) will expand on the assessments needed to optimize the configuration and it is typically followed by a decision. An alternate process (rounded corner rectangles) can be inserted late in the flow chart if a complex decision is not achieved promoting a small modification to the configuration and restarting linking back to a predetermined process. The asterisk (*) signifies a stopping point if ability to initiate a step cannot be achieved during the mapping process.

FIG. 5B shows exemplary color maps with peak-to-peak amplitude values (pV) corresponding to a representative stimulation current and electrode configuration (numbers in each row refer to contacts of the electrode array tested for the iliopsoas (IL), rectus femoris (RF), vastus lateralis (VL), medial hamstrings (MH), tibialis anterior (TA) and medial gastrocnemious (MG), muscles.

FIG. 5C shows epidural stimulation programs and resulting EMG activity of representative leg muscles during voluntary movement for knee extension and ankle dorsiflexion.

FIG. 5D shows EMG activity of representative leg muscles during stepping with no stimulation (left column) and with stimulation while assisted at different amplitudes (middle and right column), with epidural stimulation programs depicted to the right of the columns of graphs.

FIG. 5E shows EMG activity of representative leg muscles during independent stepping with stimulation, with epidural stimulation programs depicted to the right of the EMG data.

FIG. 6A depicts a representative decision-making flowchart for the selection of configurations for cardiovascular regulation. Spatio-temporal mapping for systolic blood pressure is used to identify the areas of the electrode array where blood pressure is modulated without EMG activity. In addition, the lower extremity voluntary mapping is used to examine the cathodes that promote movement. An initial configuration is represented as the data (parallelogram), decisions in the process are represented by rhombus shapes. If the response to the question is YES you continue down the flowchart if NO the flowchart will guide you to the restarting point. A predefined process (rectangles) will expand on the assessments needed to optimize the configuration and it is typically followed by a decision. An alternate process (rounded corner rectangles) can be inserted late in the flow chart if a complex decision is not achieved promoting a small modification to the configuration and restarting linking back to a predetermined process.

FIG. 6B depicts a representative cardiovascular mapping of an example of the flow of identifying the configuration, assessing the blood pressure response and modifying the configuration to result in systolic blood pressure responding to normative levels. FIG. 6B also includes color mapping of the systolic blood pressure (mmHg) corresponding to a representative stimulation current and electrode configuration (numbers in each row refer to contacts of the electrode array tested used for identifying initial cathodes for blood pressure. Corresponding EMG activity of representative leg muscles with no activity when using CV-scES.

FIG. 7A depicts a representative decision-making flowchart for the selection of stimulation parameters for maintenance of bladder continence and the initiation of voiding (collectively, “bladder compliance”). Recording MRI axial scans with high spatial resolution (3 mm slice thickness and zero gap) were used to anatomically map the lumbosacral spinal cord region relative to the paddle array. Initial selection of spinal cord stimulation parameters targeted the caudal region of the array, with activation of the striated sphincteric muscles of the perineum (external anal sphincter, EAS). Stimulation parameters targeting cardiovascular function (CV) were also integrated during bladder mapping to improve blood pressure stabilization at higher filling cystometry volumes. After the initial stimulation configuration (parallelogram) is defined, decisions in the process are represented by rectangle shapes. If the response to the question is YES the flow continues vertically down; if NO the flowchart will guide you to a restarting point to allow for small adjustments of the stimulation parameters, restarting back to a predetermined process. For FIGS. 7A-D, the following abbreviations are used: BC, bladder compliance; bpm, beats per minute; cmH₂O, centimeters of water; C, cohorts; CV, cardiovascular; EMG, electromyography in microvolts; Frequency of the stimulation (Hz); Intensity is represented in volts (V) for FIG. 7B and milliamperes (mA) for FIG. 7D; mmHg, millimeters of mercury; Pulse width is in microseconds (μs); scES, spinal cord epidural stimulation; Time in minutes (m). Cathode stimulating electrodes are represented with (−), anodes are represented with (+), and inactive electrodes are grey.

FIG. 7B, Panel 1, depicts a representative example of filling cystometry without stimulation. Note the rapid rise in detrusor pressure during filling (upper graph) and a concomitant elevation of blood pressure (lower graph), timed to each bladder contraction. Sensations of bladder fullness (labeled vertical lines) coincide with detrusor overactivity and increased EMG activity (middle graph) at low bladder capacity. Filling cystometry ceased due to the loss of continence (labeled vertical lines). FIG. 7B, Panel 2, depicts a representative example of mapping for bladder compliance (BC) demonstrating increased bladder capacity and reduced detrusor pressure with stimulation. FIG. 7B, Panel 3, depicts a representative example of mapping for bladder compliance (BC) demonstrating increased bladder capacity and reduced detrusor pressure with stimulation, and additional stimulation to regulate blood pressure. Maintenance of both normal systolic blood pressure (target <120 mmHg) and standard bladder filling pressure (<10 cmH₂O) during cystometry required both BC-scES and CV-scES cohorts as shown by comparing Panels 1, 2, and 3.

FIG. 7C depicts a representative decision-making flowchart for the selection of stimulation parameters for the initiation of bladder voiding. In case the mapping process does not result in the targeted functional outcome (initiation of voluntary voiding), stimulation configuration may be determined at the flow-chart level identified with the asterisk (*). Abbreviations are the same as those in FIG. 7A.

FIG. 7D, Panel 1, depicts an exemplary urological profile during the voiding phase revealing detrusor contractions not sufficient to decompress the EAS activity during the void attempt without the use of scES. FIG. 7D, Panel 2, depicts an exemplary bladder voiding (BV) phase trace with stimulation demonstrating a more synchronized detrusor-sphincter relationship resulting in the generation of a bladder contraction from a stable filling pressure (<10 cmH₂O) sufficient initiate voiding. Note that a uroflow was not tested in the mapping environment and the EMG displayed reflects the linear envelope of the signal. A blood pressure response occurred beginning with the void attempt (start indicated with the first vertical line), followed by a return to baseline values once voiding initiated (indicated with the second vertical line), likely reflective of the participants' effort to empty.

FIG. 8 depicts an exemplary schematic representation of participant information and experimental setup for scTS. Panel (a) is MRI showing the region of the spinal injury. The injury spans the T8-T11 (vert) levels. Panel (b) shows electrode placement for scTS. Cathode/Anode pairs. Panel (c) depicts participant positioning in the Gravity Neutral Device. Both legs of the participant are suspended to minimize the effect of limb weight on limb movement. Panel (d) depicts the participant in the body weight-support treadmill with trainers. The drawing displays hand positioning of the trainer to facilitate stepping during training.

FIG. 9 depicts representative computational simulations in an exemplary virtual phantom showing differential distribution of charge density after scTS at different stimulation sites. Specifically, Panel (a) depicts placement of the cathodes and their respective anodes on the virtual phantom; Panel (b) depicts a sagittal view of the current density for each stimulation site; and Panel (c) depicts current density at the dorsal surface of the spinal cord and spinal nerves for each stimulation site.

FIG. 10 depicts representative stick diagrams and cyclograms showing hip, knee, and ankle joints during voluntary stepping in a non-weight bearing setting across baseline, after nWBT, and after WBT without scTS (Panels a, b, and c) and with scTS (Panels d, e, f).

FIG. 11A depicts exemplary EMG traces of the right leg collected during stepping assessments using scTS in Body Weight Supported Treadmill before weight-bearing training (nWBT) and after weight-bearing training (WBT).

FIG. 11B depicts total power and mean frequency of the proximal (left) and distal (right) muscles collected during an exemplary stepping assessments using scTS in Body Weight Supported Treadmill before and after WBT.

FIG. 11C depicts an exemplary normalized integrated EMG between agonist and antagonist muscle pairs to show coordination between muscle pairs in both left and right legs before and after WBT.

FIG. 11D is a representative graph depicting a representative change in relative Pearson correlation between muscles sets after WBT. A positive change indicates improvement in muscle coordination, while a negative change indicates decline in coordination.

FIG. 12 includes Panels (a) and (b) depicting supralesional modulation of MEPs using scTS after conditioning through non-specific cervico-lumbar pathways, propriospinal system, and reticulospinal pathways before training (baseline), after non-weight bearing training (nWBT), and weight-bearing training (WBT). Panel (a) depicts exemplary motor responses of the right tibialis anterior (R TA) muscle without conditioning (grey) and with supralesional conditioning. Panel (b) depicts a representative percent change of the area under the curve of the spinal evoked responses after modulation when compared to non-conditioned pulses for all muscles of the right leg.

FIG. 13 includes Panels (a) and (b) depicting voluntary modulation of motor evoked potentials using scTS during voluntary tasks before and after treadmill training. Panel (a) depicts exemplary spinal cord evoked motor potentials of the main muscle involved in each voluntary task. Panel (b) depicts a representative difference of the area under the curve compared to control pulses across all muscles for each voluntary task.

FIG. 14 includes Panels (A) and (B) depicting scatter plots of the pressure-volume measurements obtained during urodynamics without scES (open squares) and from mapping with BC-scES (filled in squares) in subjects using intermittent catheterization (Panel A), n=4, and in those using suprapubic catheters (Panel B), n=3. Range of blood pressure responses at maximum capacity are superimposed above each plot. Transparent background ellipses show the trends of the measurement distributions for each participant. Vertical and horizontal lines indicate normative thresholds for minimum bladder capacity and maximum detrusor pressure, respectively. (BC-scES, bladder compliance spinal cord epidural stimulation; cmH2O, centimeters of water; ml, milliliters; mmHg, millimeters of mercury).

FIG. 15 includes Panels (A) and (B) depicting improvement in bladder compliance using targeted scES parameters (BC-scES). (Panel A) depicts an example of detrusor pressure (black, top panel) and bladder capacity (red, top panel), sphincter EMG (μV, middle panel), blood pressure (mmHg, black, systolic—top line, diastolic-bottom line, lower panel) and heartrate (red, lower panel) in the absence of scES in a participant with chronic SCI (B24); Note the detrusor overactivity with incontinence at low capacity, and a simultaneous rise in systolic blood pressure. Panel (B) depicts similar data for the same participant using BC-scES and parameters adjusted for bladder compliance. Maintenance of bladder compliance (increased bladder capacity without a change in detrusor pressure in response to bladder filling) was intensity (V, gray bar) dependent and subject specific. Note the tonic sphincter EMG activity to facilitate urinary continence.

FIG. 16 includes Panels A, B, and C illustrating a comparison of bladder capacity, detrusor pressure and systolic blood pressure without scES relative to optimized BC-scES parameters for representative participants intermittently catheterizing, n=4; and Panels D, E, and F for representative participants with a suprapubic catheter, n=3. As shown, BC-scES mapping significantly improved (reduced) detrusor pressure and systolic blood pressure at maximum capacity in those using intermittent catheterization and detrusor pressure in the suprapubic group.

FIG. 17A displays an example of detrusor pressure (upper chart) and sphincter EMG (μv, lower chart) in the absence of scES in an individual with chronic SCI (B21).

FIG. 17B displays representative cystometry recording in the same individual as in FIG. 17A using BV-scES and parameters adjusted for void initiation; Voiding initiation was intensity (pink/red bar) dependent and subject specific. Note the rise in detrusor pressure timed with relaxation of sphincter EMG activity and a return of detrusor pressure to baseline.

FIG. 17C is a chart illustrating representative effective and non-effective BV-scES parameters for promoting volitional voiding during urodynamics mapping sessions for an individual with chronic SCI (B07). Initiating a void occurred only in the presence of optimized BV-scES. Reflexive leaks are indicated as involuntary. Light gray indicates the voiding efficiency (VE) for a single leak/void and dark gray indicates total voiding efficiency for a mapping session when multiple void attempts were possible. (VE=[Void Amount/Void+Residual]*100).

FIG. 18 includes Panels A and B containing exemplary color map plots of the amount of electric charge delivered per second using scES parameters, i.e. intensity, frequency, pulse width and electrode combinations, that resulted in improved bladder compliance outcomes (Panel A) and initiating voiding (Panel B) for each participant (x-axis) and the levels of the spinal cord that were directly targeted by the stimulation (y-axis). FIG. 18, Panel C, depicts an example of MRI-based 3D model of the spinal cord at the lumbosacral enlargement and the location of scES paddle array with respect to the spinal cord. Distribution of electric current density (A/m²) is highlighted with the color map. Simulations are performed using the Sim4Life platform. (BC, bladder compliance; BV, bladder voiding; L, lumbar; S, sacral; scES, spinal cord epidural stimulation; T, thoracic.

### DETAILED DESCRIPTION OF THE PREFERRED EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

A “motor pool” comprises all or substantially all individual motor neurons that innervate a single muscle.

Spinal cord epidural stimulation (“scES”) refers to stimulation of the spinal cord by electrical stimulation delivered by an implanted neurostimulator.

Spinal cord transcutaneous stimulation (“scTS”) refers to stimulation of the spinal cord by electrical stimulation delivered non-invasively by a neurostimulator applied to skin.

The terms scES or scTS may be preceded by a two letter abbreviation of the physiological function targeted by the stimulation, e.g., UE-scTS is spinal cord transcutaneous stimulation targeting the upper extremities; CV-scES is spinal cord epidural stimulation targeting the cardiovascular system.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount is meant to encompass variations of ±10% of the most precise digit in the value or amount (e.g., “about 1” refers to 0.9 to 1.1, “about 1.1” refers to 1.09 to 1.11, etc.).

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The presently-disclosed subject matter relates to systems and methods for improvement of physiological function in an individual with a spinal cord injury via electrical stimulation of the spinal cord. Such systems and methods include (i) anatomical specificity to target the appropriate region of the spinal cord for stimulation, (ii) electrical specificity to provide the appropriate stimulation by delivery method, frequency, pulse duration, and other factors, and (iii) physiological specificity to evoke, suppress, increase, or decrease a specific physiological result. In some embodiments, multiple regions of the spinal cord may be subject to electrical stimulation for concurrent improvement of multiple physiological functions. The physiological functions to be improved include at least one of lower extremity voluntary motor movement, posture, locomotion, upper extremity voluntary motor movement, bladder function, bowel function, cardiovascular function, and respiratory function.

Overview of anatomical specificity, electrical specificity, and physiological specificity for systems and methods for improvement of physiological function in an individual with a spinal cord injury via electrical stimulation of the spinal cord.

Locomotion and Posture

Epidural and transcutaneous spinal cord neuromodulation can both be specifically configured by electrode selection and placement, pulse width, frequency, amplitude, and other factors to promote integration of sensory feedback and supraspinal control through spinal networks with sufficient excitability.

TABLE 1
Specificity for Locomotion and Posture Improvement
by Spinal Cord Stimulation

| Specificity | Feature |
|---|---|
| Anatomical Specificity | Targeting different motor pools |
| Electrical Specificity | Selection of anodes and cathodes |
| | Frequency |
| | Pulse duration |
| | Amplitude |
| | Pulse width |
| Physiological Specificity | To increase/decrease excitability |
| | of motor system |
| | To increase supraspinal integration (combination |
| | of scES + scTS at cervical levels) |
| | To increase sensory integration |
| | To maintain other systems as stable (blood |
| | pressure, bladder, etc.) |
| Synchronization of | Increase brain-spinal connectomes |
| scES and scTS | Increase coordination during stepping |
| | Facilitate integration during complex tasks |

Upper Extremity

Spinal cord neuromodulation can be specifically configured by electrode selection and placement, pulse width, frequency, amplitude, and other factors via transcutaneously or epidurally delivered electrical spinal cord neuromodulation for upper extremity voluntary motor movement (UE-scTS or UE-scES).

Bladder

Epidural or transcutaneous spinal cord neuromodulation can be specifically configured by electrode selection and placement, pulse width, frequency, amplitude, and other factors to promote integration of sensory feedback and supraspinal control through spinal networks with sufficient excitability to improve bladder storage and emptying.

TABLE 2
Specificity Bladder Improvement by Spinal Cord Stimulation

| Specificity | Feature |
|---|---|
| Anatomical Specificity | Targeting sacral motor pool |
| | Targeting lumbar coordinating center |
| Electrical Specificity | Selection of anodes and cathodes |
| | Frequency |
| | Pulse duration |
| | Amplitude |
| | Pulse width |
| Physiological Specificity | To increase/decrease excitability of the |
| | sympathetic and parasympathetic autonomic |
| | nervous systems |
| | To increase supraspinal integration (combination |
| | of intent with sensations of bladder fullness) |
| | To increase sensory integration |
| | To maintain other systems as stable (blood |
| | pressure, etc.) |

Bowel

Epidural or transcutaneous spinal cord neuromodulation can be specifically configured by electrode selection, pulse width, frequency, amplitude, and other factors to promote integration of sensory feedback and supraspinal control through spinal networks with sufficient excitability to improve bowel motility, storage and emptying.

TABLE 3
Specificity Bowel Improvement by Spinal Cord Stimulation

| Specificity | Feature |
|---|---|
| Anatomical Specificity | Targeting sacral motor pool |
| | Targeting lumbar coordinating center |
| Electrical Specificity | Selection of anodes and cathodes |
| | Frequency |
| | Pulse duration |
| | Amplitude |
| | Pulse width |
| Physiological Specificity | To increase/decrease excitability of the |
| | sympathetic and parasympathetic autonomic |
| | nervous systems |
| | To increase supraspinal integration (combination |
| | of intent with sensations of rectal fullness) |
| | To increase sensory integration |
| | To maintain other systems as stable |

Cardiovascular Function

Transcutaneously or epidurally delivered electrical spinal cord neuromodulation can be specifically configured by electrode selection, pulse width, frequency, amplitude, and other factors for improving cardiovascular function (CV-scTS or CV-scES).

TABLE 4
Specificity Cardiovascular Function Improvement by Spinal Cord Stimulation

| Specificity | Feature |
|---|---|
| Anatomical Specificity | Above level of spinal cord lesion |
| | At site of spinal cord lesion |
| | Below level of spinal cord lesion |
| Electrical Specificity | Number of channels |
| | Modulation (modulated or non-modulated) |
| | Phasing (monopolar or bi-polar) |
| | Frequency |
| | Pulse duration |
| | Amplitude |
| | Pulse width |
| Physiological Specificity | To increase/decrease excitability of sympathetic |
| | system |
| | To increase/decrease excitability of |
| | parasympathetic system |
| | To increase/decrease systemic arterial Blood |
| | Pressure (BP) |
| Synchronization with | Close-loop synchronization with real-time BP |
| BP changes | changes |
| | Support during orthostatic event |
| | Support during autonomic dysreflexia event |
| Synchronization with | Autonomic pre-conditioning before |
| cardiovascular training | Cardiovascular Training |
| interventions | (based on Valsalva-like maneuvers) |
| | To increase/decrease excitability of spinal motor |
| | networks for cardiovascular function during |
| | before Cardiovascular Training |
| | To increase/decrease BP during relaxation used |
| | as training intervention |

Respiratory Function

Specifically configured transcutaneously or epidurally delivered electrical spinal cord neuromodulation configured by electrode selection, pulse width, frequency, amplitude, and other factors for improving respiratory function.

TABLE 5
Specificity Respiratory Function Improvement by Spinal Cord Stimulation

| Specificity | Feature |
|---|---|
| Anatomical Specificity | Above level of spinal cord lesion |
| | At site of spinal cord lesion |
| | Below level of spinal cord lesion |
| Electrical Specificity | Number of channels |
| | Modulation (modulated or non-modulated) |
| | Phasing (monopolar or bi-polar) |
| | Frequency |
| | Pulse duration |
| | Amplitude |
| | Pulse width |
| | Anode and cathode positioning |
| Physiological Specificity | To increase/decrease excitability of spinal motor |
| | networks for respiration |
| | To increase/decrease excitability of autonomic |
| | networks for respiration |
| | To support/evoke inspiratory phase |
| | To support/evoke expiratory phase |
| | To support cough |
| | To support swallow |
| | To support cough during swallow |
| Synchronization with | To increase/decrease excitability of spinal motor |
| ventilatory support | networks for respiration |
| | To support/evoke inspiratory phase |
| | To support/evoke expiratory phase |
| | To support/evoke cough during expiratory phase |
| Synchronization with | Spinal network pre-conditioning before |
| Respiratory Training | Respiratory Training |
| intervention | To increase/decrease excitability of spinal motor |
| | networks for respiration during Respiratory |
| | Training |
| | Support of specific breathing phase |
| | To support inspiratory phase |
| | To support expiratory phase |

In order to provide spinal cord stimulation targeted to one or more, two or more, or three or more, or four or more physiological functions, scES is functionally configured by the following process: 1) Neuroanatomical 3D spinal cord reconstruction model and intra-operative evoked potentials are used to inform electrode lead placement; 2) spatial-temporal electrophysiological mapping is used to inform motor and autonomic mapping; and 3) task specific motor mapping (leg movements, standing and stepping) and autonomic mapping (blood pressure regulation, bladder capacity and initiation of bladder void). Immediate integrated movement and physiological responses to scES indicates the sophistication of the human lumbosacral spinal circuitry with the critical role for ongoing regulation of motor and autonomic function.

Subhead 1: Neuroanatomical 3D Spinal Cord Reconstruction Model and Surgical Implantation

Prior to surgery, participants undergo an MRI that includes obtaining sequential axial T2-Turbo Spin echo images from the T10-S1 vertebral with high spatial resolution (3 mm axial thickness and zero gap) using a Siemens 3.0 Tesla MAGNETOM Skyra with Turbo Spin Echo (Siemens Medical Solutions, Malvern, PA). A 1.5 Tesla (General Electric, Milwaukee, WI) is used in cases when hardware implants contraindicate higher magnet strengths. We observed considerable variability among individuals regarding the location of conus termination with respect to vertebrae ranging from vertebrae T12 to L2. The length of the lumbosacral spinal cord also varies across individuals and therefore the location of spinal cord levels L1-S1 also varies with respect to the vertebral bodies. The neuroanatomical 3D spinal cord reconstruction from sequential axial images that includes dorsal root tracing provides a more precise determination of the neuroanatomical characteristics of the spinal cord. During surgery, the objective is to place the electrode paddle midline of the vertebral column over the L1-S1 spinal cord levels while covering a significant area of the lumbosacral enlargement, and more specifically, the spinal cord level(s) the stimulation of which promotes one or more desired physiological functions.

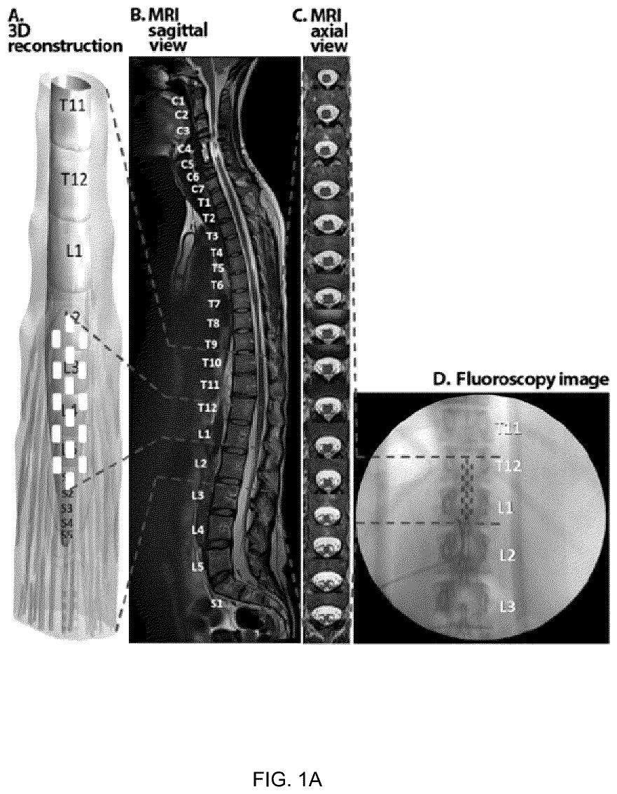

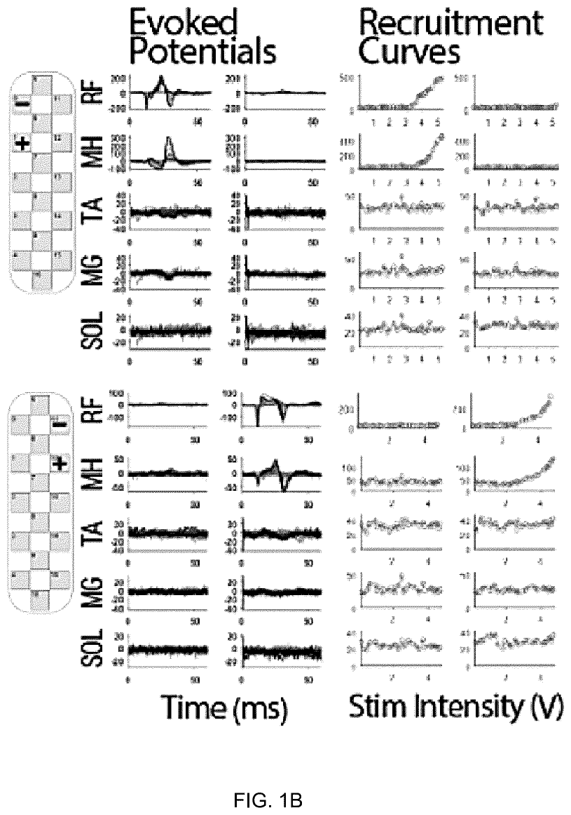

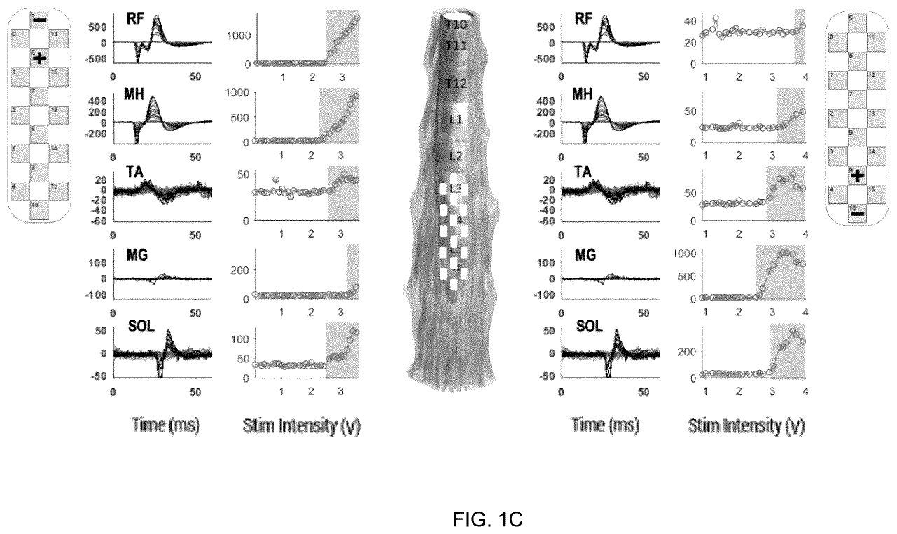

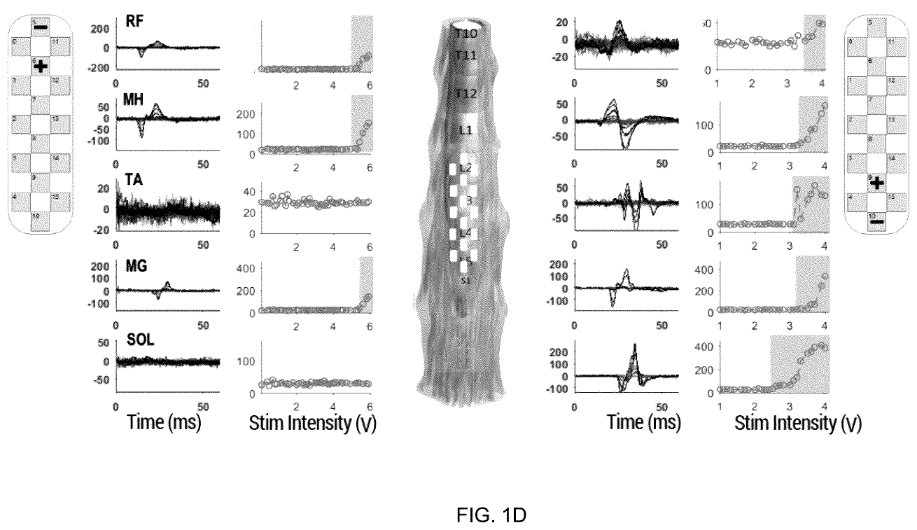

A 3D visual model of the spinal cord is generated from ROI tracing (Mango software, Research Imaging Institute, UTHSCSA) of the circumference of the spinal cord, the dura border and dorsal roots. The L1-S1 spinal cord levels at the lumbosacral enlargement were identified by performing nerve root tracing from the sequential axial images from the locations where the nerve roots exit the vertebrae and emerge from the spinal cord and then delineated on the 3D visual model. The Neuroanatomical 3D reconstruction model was generated (FIG. 1A, panels A, B and C) from quantitatively integrating the sagittal images of the vertebrae, the known dimensions of the scES paddle (Medtronic Specify® 5-6-5 lead, 46.5 mm) and the location of the nerve roots emerging from the spinal cord.

Prior to the implant, possible positions of the electrode paddle are projected onto the MRI-based 3D model and corresponding percentages of lumbosacral enlargement coverage are calculated. These information is used in the identification of the level of surgical laminotomy. In most individuals the length of the lumbosacral enlargement is greater than the total length of the electrode contacts in the scES paddle and therefore the placement is informed by the knowledge of those lumbosacral spinal cord levels that have been most successful in the recovery of deficits. Following the initial positioning of the electrode array, intra-operative evoked potentials are elicited, and muscle activation thresholds and amplitude response curves are evaluated in real-time. Mid-line and rostral-caudal positioning of the paddle is refined as shown in FIGS. 1C and 1D. Fluoroscopy images of the selected placement are recorded (see FIG. 1A, panel D) and used to recalculate the spinal cord coverage by the analytical team.

Subhead 2: Spatio-Temporal Mapping

Spatio-temporal electrophysiological mapping is the first step in identifying the spatial relationship between stimulation site and motor evoked responses in specific muscles. Exemplary interoperative spatio-temporal mapping is shown in FIG. 1B with overlaid evoked potentials and recruitment curves of muscle responses to the stimulation with various electrode combinations from the scES paddle array, with EMG recorded from rectus femoris (RF), medial hamstrings (MH), tibialis anterior (TA), medial gastrocnemius (MG), soleus (SOL). Similarly, we identify threshold and temporal relationships of lower extremity muscles during the mapping process. Spatio-temporal mapping typically occurs 2-3 weeks post implant over 2-3 days and is performed while the individual rests in a supine position. Electromyography (EMG) of 8 lower extremity muscles (soleus, medial gastrocnemious, tibialis anterior, medial hamstrings, vastus lateralis, rectus femoris, gluteus maximus and iliopsoas) are recorded bilaterally during the session. In addition, surface electrodes are placed symmetrically over the paraspinal muscle adjacent to the electrode array incision in order to detect stimulation artifacts, which define the onset of each stimulation pulse. Continuous beat-to-beat blood pressure is also monitored throughout the mapping session.

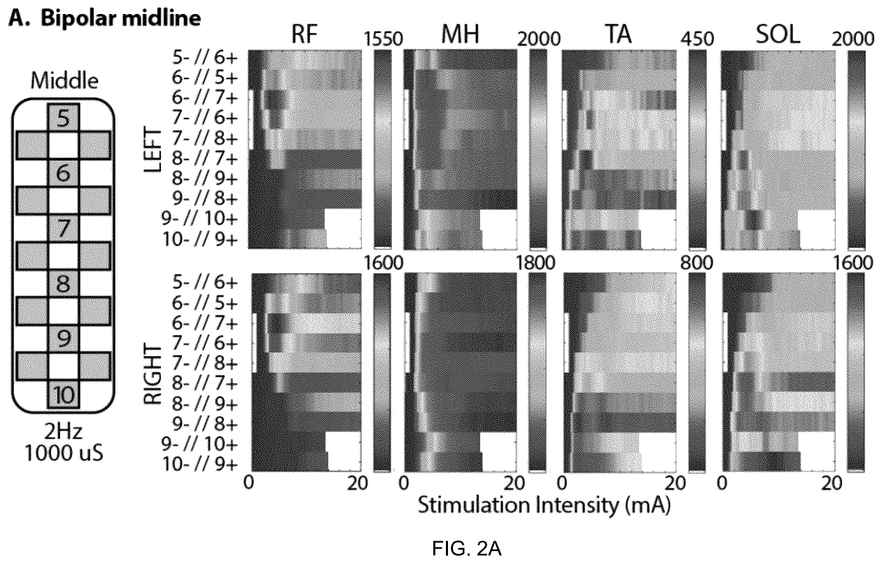

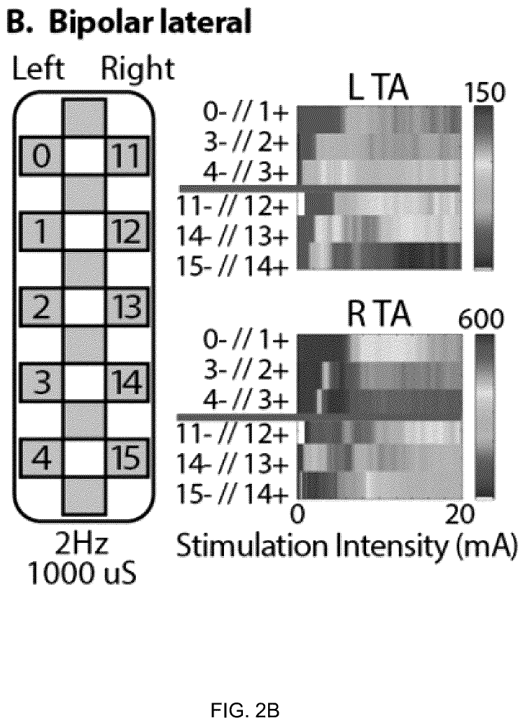

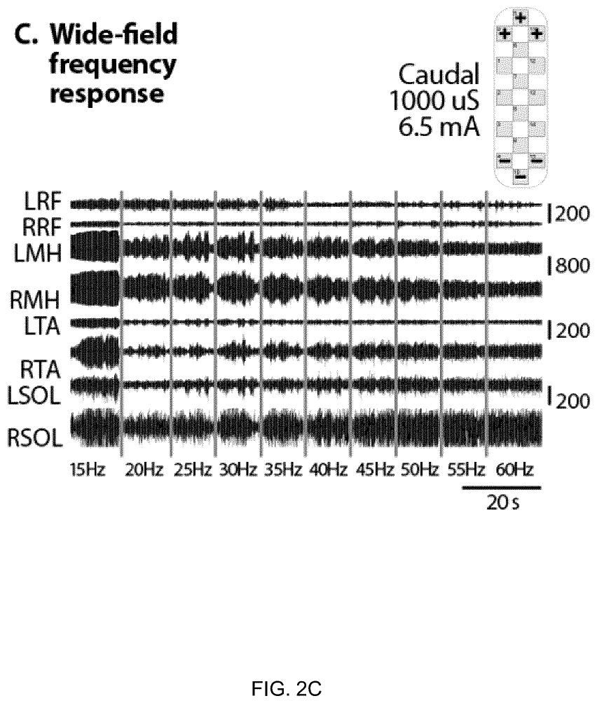

Spatio-temporal mapping is performed using a pulse-width of 1000 μsec and consists of 4 sections: (1) bipolar midline amplitude response; (2) wide-field amplitude response; (3) wide-field frequency response; (4) bipolar lateral amplitude response. Ten bipolar combinations are tested along the middle column during the initial amplitude response with a stimulation frequency of 2 Hz (FIG. 2A). Adjacent anode and cathode electrodes are selected in the middle column, combinations are randomized for each participants. Stimulation starts at 0.1 mA, or the highest pre-threshold amplitude, and increased by 0.1 mA until all assessed muscles have reached motor threshold. The amplitude response continues at increments of 0.5 mA until all muscles have reached a plateau in the peak-to-peak amplitude, maximum stimulation is reached, the blood pressure response has increased over 160 mmHg systolic, or the participant requests to stop. The bipolar lateral amplitude response follows the same process, except 3 combinations are chosen for each side to represent a rostral, middle and caudal location (FIG. 2B). Similarly, the wide-field amplitude response is performed with the top three electrodes (0,5,11) and bottom three electrodes (4,10,15) selected as cathodes and anodes and both rostral and caudal configurations tested. For the wide-field configurations, stimulation amplitude starts at 0.3 mA and continues in increments of 0.3 mA; stopping can occur for the same reasons as detailed above. A comparison of certain components of the above detailed mapping is also performed with a pulse width of 450 μsec. This included selected midline rostral, middle and caudal bipolar configurations and the rostral and caudal wide-field configurations.

Frequency responses are performed using the wide-field configurations at two different stimulation amplitudes. The first stimulation amplitude, considered low, is selected following the onset of all muscles as determined in the 2 Hz wide-field amplitude response. The amplitude will be near the lower third of the recruitment curve slope. A second stimulation amplitude, considered high, represents a range on the top ⅓ of the slope, close to the peak-to-peak plateau. We test frequencies from 15 Hz through 100 Hz in increments of 5 Hz, each frequency is assessed for 10 seconds (FIG. 2C). Elevation in blood pressure and discomfort are typical reasons that 100 Hz is not reached and can be most common in the high amplitudes. The high amplitude response is repeated with a 450 μsec pulse width. The frequency response data is valuable in identifying activation patterns (locomotor, bursting and tonic) through the various frequencies.

In addition, a 30 Hz amplitude response is performed at selected rostral, middle and caudal bipolar configurations as well as wide-field configurations. This additional amplitude response are performed at both 1000 μsec and 450 μsec pulse widths. Identification of bursting patterns by location, amplitude and pulse width will provide key information for functional mapping of voluntary movement, standing and stepping.

All 2 Hz data is analyzed and visualized using previously described methods. Data can be easily visualized to understand the relationship between muscles and electrode selection (FIGS. 2A and 2B). These color maps provide key information about individual responses, in addition, we can learn from similar activation patterns among individuals and reduce the gap between spatio-temporal mapping and functional mapping.

Subhead 3: Selection of Electrode Configuration for Voluntary Movement

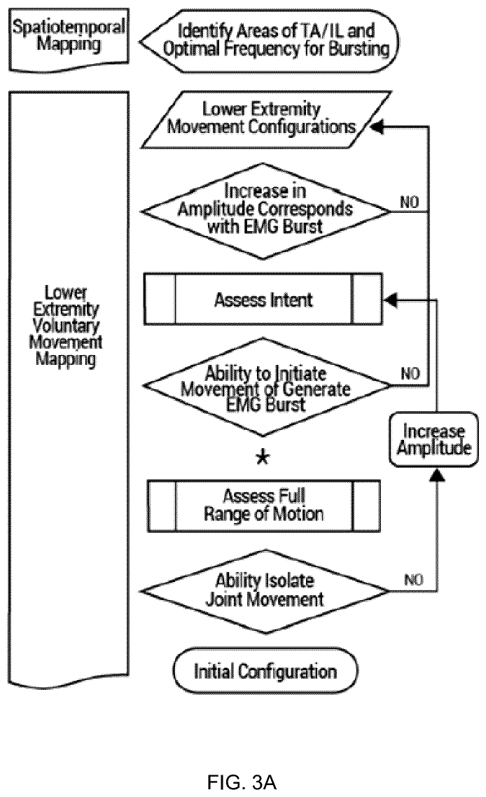

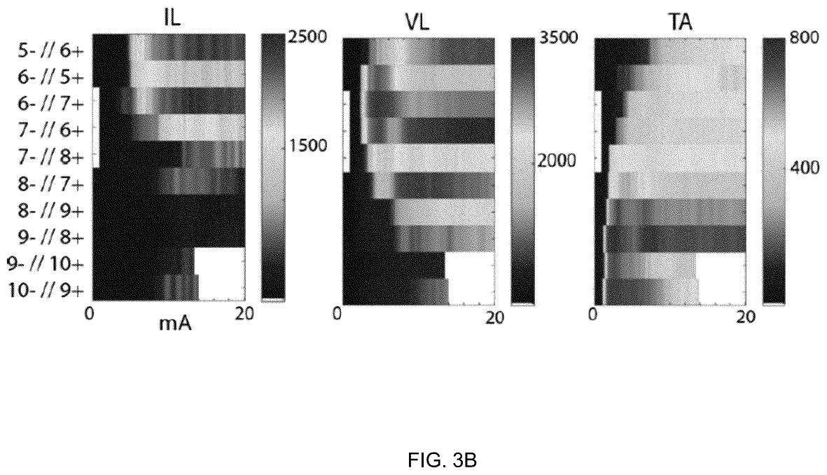

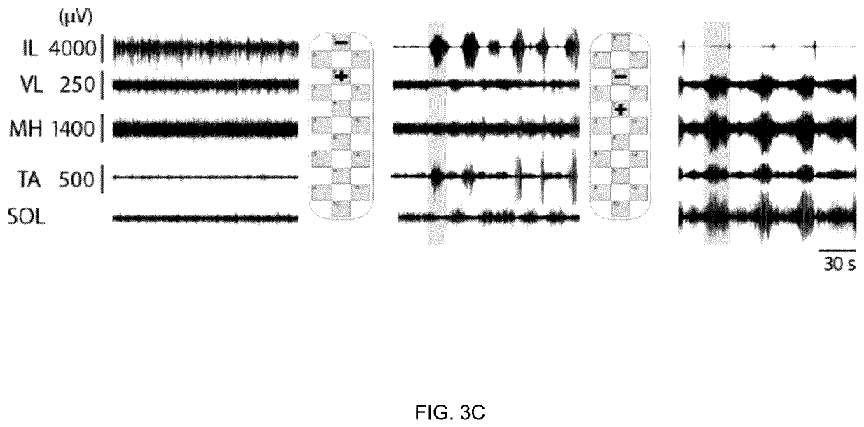

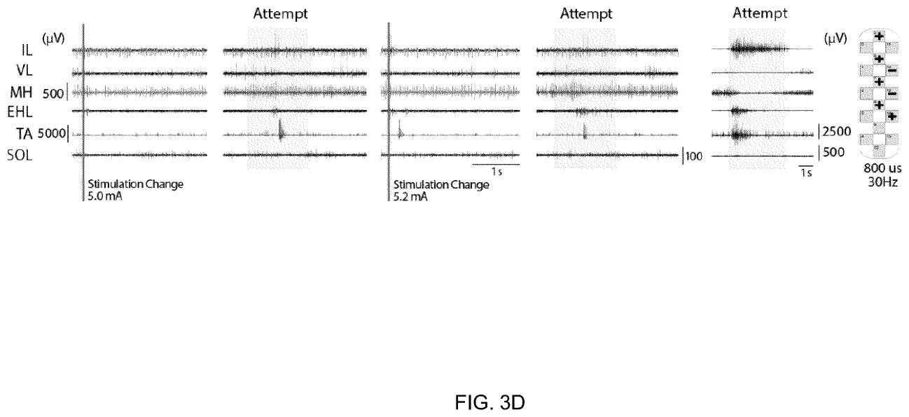

Voluntary movement requires a configuration that increases or decreases the excitability of a specific area of the spinal cord but remains sub-motor threshold. A sub-motor threshold configuration optimized for a specific action allows the integration of supraspinal signals by the spinal networks resulting in the intended movement. Evaluation of the color maps generated from the spatio-temporal mapping will provide a starting point for distribution of cathodes and anodes. Points to consider when evaluating the color maps are 1) stimulation amplitude for motor threshold relative to cathode and anode location; 2) peak to peak changes relative to stimulation amplitude and stimulation amplitude at which maximum peak to peak spread is achieved; 3) relationship for motor threshold and maximum amplitude of peak to peak between antagonistic muscles; 4) left to right differences on the same muscle. An important aspect to consider is the activation of the tibialis anterior as a key muscle not only for the execution of ankle dorsiflexion but as an indicator for hip flexion given its synchronicity with the iliopsoas. FIG. 3A provides a decision making flowchart to guide investigators in mapping for voluntary movement. In addition to the 2 Hz spatio-temporal mapping analysis, the frequency response data and 30 Hz spatio-temporal mapping data are used to understand co-activation and alternation patterns of specific muscle groups and the frequencies that promote bursting and alternation patterns (FIGS. 3B and C).

Assessment of configurations for voluntary leg movement initially take place during supine or semi-reclined position (15-30 degrees). The initial configurations are tested by slowly incrementing stimulation amplitude and asking the individual to perform the intended action. Stimulation amplitude will continue to be increased until the individual is able to perform the movement or activation of the agonist is induced by the stimulation, therefore being supra-motor threshold. Stimulation amplitude must remain sub-motor threshold for the agonist and synergistic muscle groups, therefore promoting integration of residual input and sensory information as opposed to inducing the movement.

Multiple attempts might be required for the individual to decipher how to interact with sub-motor threshold stimulation. In most cases, individuals have been injured for over 2 years without attempting to perform voluntary movement of their paralyzed lower extremities. It is imperative for the investigator leading the assessment to guide the individuals based on electrophysiological data and suggesting strategies for accessing the appropriate networks. If a change in stimulation amplitude induces a small single movement or EMG burst on the agonist muscle, the location of anode and cathode distribution is appropriate and stimulation amplitude and frequency remain to be adjusted (FIG. 3D). Early in the mapping phase small changes to electrode selection might have a large impact on the ability of the individual to perform the desired movement. This might be the result of the individual's learning to integrate the stimulation with the supraspinal intent as well as focusing the stimulation towards the appropriate motor pools.

Subhead 4: Selection of Stimulation Parameters for Standing

Dedicated guidelines were developed to determine the subset of electrode configurations to be tested for facilitating standing. These guidelines are based on the available literature and on “spatio-temporal mapping” assessments performed in supine position.

Earlier evidence of lower limb extension pattern generation after SCI. Earlier research showed that lumbosacral scES has the potential to access the human spinal circuitry and induce the generation of lower limb motor patterns after complete SCI. In particular, high stimulation amplitudes delivered at frequencies between 5 and 15 Hz were able to drive lower limb tonic extension in SCI participants laying supine. Also, electrode configurations with the cathode (active electrode) positioned in the caudal area of the lumbosacral spinal cord, and more caudal than the anode, were considered more effective for generating lower limb extensor pattern, primarily because lower stimulation amplitude was required to elicit that pattern. While we do not implement this stimulation approach (i.e. using high-intensity and low frequency epidural stimulation to drive lower limb extension) to promote standing motor recovery, it is important to considered that the caudal area of the lumbosacral spinal cord appears more responsive to scES for eliciting extension pattern generation.

Topographical organization of the activation pattern facilitated by scES.

Stimulation site and electrode configuration have important implications for topographical organization of the activation pattern facilitated by scES. For example, electrode fields more focused on the caudal portion of the electrode array (i.e. spinal cord segments L5-S1) can be selected to promote activation of distal muscles' motoneuron pools, while electrode fields focused on the rostral portion of the array (i.e. spinal cord segments L1-L2) can be activated to promote activation of proximal muscles' motoneuron pools. Also, in case of activation differences between left and right lower limb, active electrodes can be unbalanced between lateral columns of the electrode array, as the lateral placement of the epidural stimulation electrodes with respect to the spinal cord midline was shown to promote motor responses in muscles ipsilateral to the stimulation.

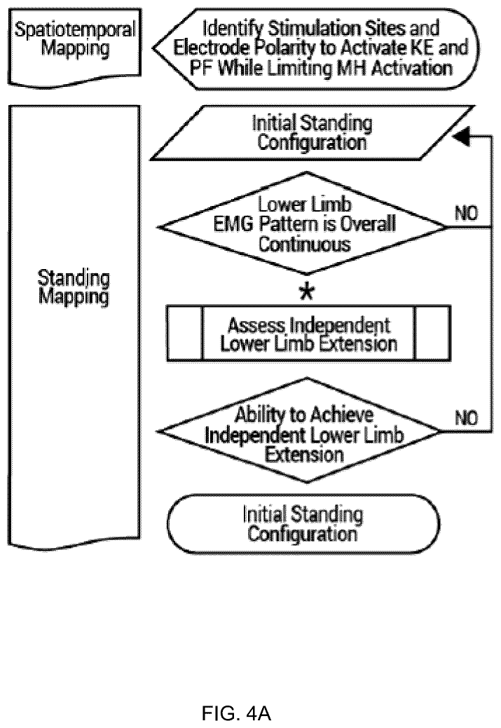

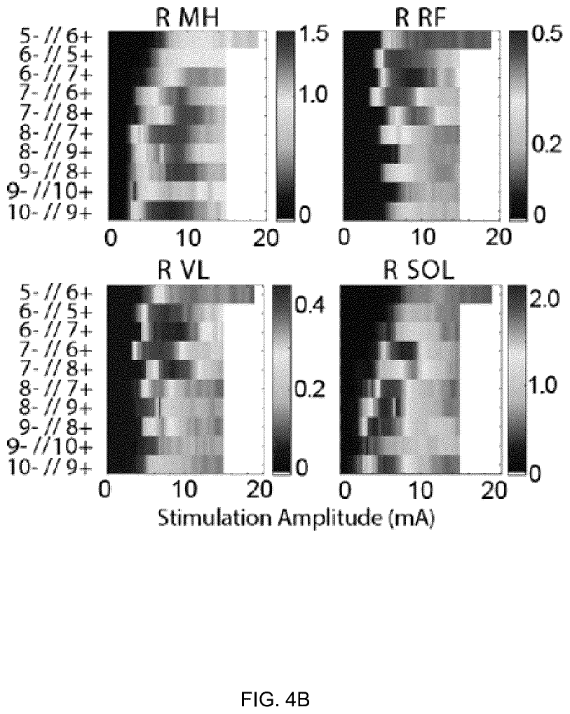

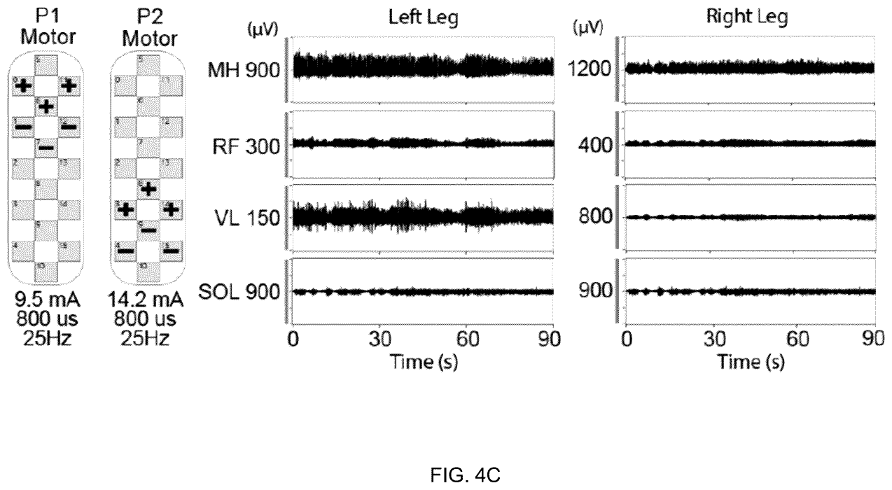

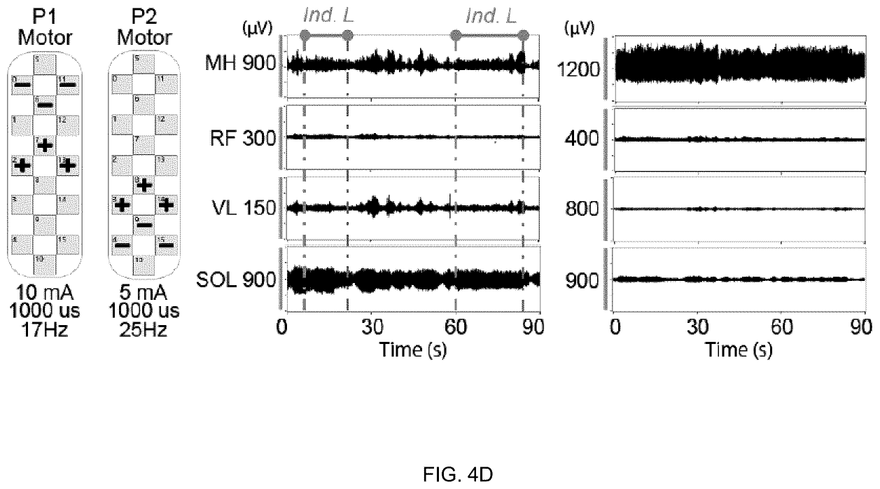

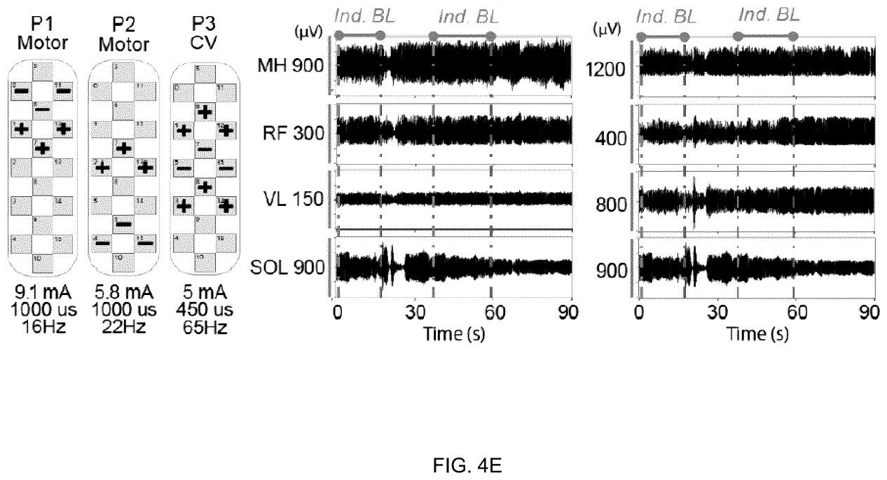

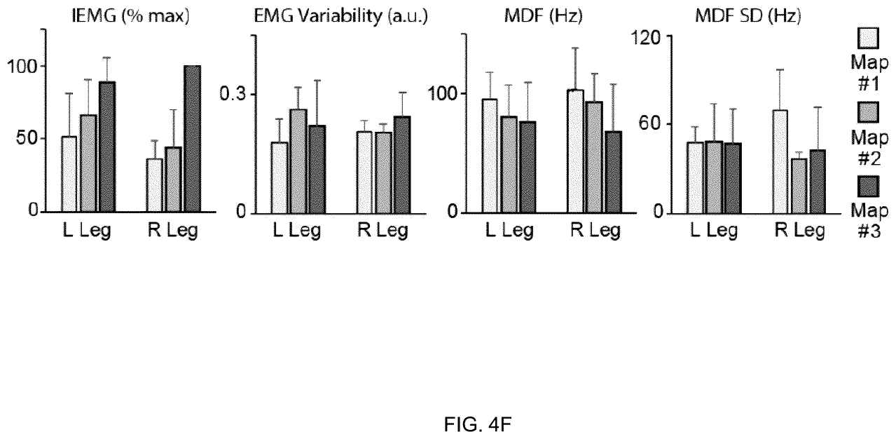

Individualized maps of motor pools activation are determined during “spatio-temporal mapping” assessments that consider, among others, muscle activation responses to different localized two-electrode configurations (FIGS. 4B and C), using 2 Hz stimulation frequency and increasing amplitude, with the research participants in supine position. Stimulation amplitude and evoked potentials peak-to-peak amplitude for each electrode configuration tested are then reported as color maps for each investigated muscle (FIG. 4C). This information is used to adjust the position of cathodes in order to target primarily extensors muscle groups while limiting the activation of primary flexor muscles.

Selection of scES amplitude and frequency to facilitate standing. Earlier studies showed that high scES amplitude applied to SCI individuals in supine position can elicit lower limb tonic extension or locomotor-like patterns, depending of the stimulation frequency applied (i.e. 5-15 Hz or 25-60 Hz, respectively). However, we successfully facilitated standing applying high stimulation frequencies (i.e. 25 to 60 Hz) at near-motor threshold stimulation amplitudes that did not elicit directly lower limb movements in sitting. In particular, the application of higher stimulation frequencies can favor the integration of afferent input and residual supraspinal input through the greater involvement of interneurons, and results in a more physiological (i.e. non-pulsatile) muscle contraction. This is consistent with our approach of using scES for enabling the spinal circuitry to integrate weight bearing-standing related sensory information, and possibly residual descending input, to generate motor patterns effective for standing, rather than using scES to drive an activation pattern. Hence, scES for standing is initially delivered at a near-motor threshold stimulation amplitude that does not elicit directly lower limb movements in sitting. Stimulation amplitude and frequency are then synergically modulated during standing in order to identify the higher stimulation frequency that can elicit a continuous (non-rhythmic) EMG pattern effective to bear body weight.

The decision-making process of selection of spinal cord stimulation parameters to facilitate standing is depicted in FIG. 4A, and exemplary outcomes for a representative research participant with motor complete SCI and cervical level of injury (B38) are reported in FIG. 4C-F. Individualized activation maps (FIG. 4B) suggested to focus stimulation on the spinal cord segment L3, corresponding to the rostral part of the array (6−/7+) to engage knee extensors (RF and VL) while limiting the activation of knee flexors (MH). Electrode configuration of P1 (FIG. 4C-E) was selected to facilitate this motor outcome. Cathodes were initially placed more caudally than anodes (FIG. 4C), and then reversed (FIG. 4D-E) as suggested by the higher activation promoted for VL and RF in supine by the anode placed more caudally than the cathode (see ‘6−/7+’ versus ‘7−/6+’, FIG. 4B). Electrode field of P2 (FIG. 4D-E) initially targeted the spinal cord segments L4-L5 to facilitate the activation of plantar flexor muscles as suggested by the individualized activation maps (FIG. 4B, SOL, electrodes ‘9−/8+’), and was subsequently extended (FIG. 4E), covering L3-L5 spinal segments, to further contribute to the activation of more proximal motor pools. Stimulation frequency of P1 and P2 were slightly decreased, and pulse width increased, throughout the standing map process (FIG. 4D-F) to contribute to facilitate extension pattern and higher muscle activation. Along the process of standing stimulation mapping, a stimulation program targeting the increase in blood pressure (P3, FIG. 4E) was added because of the persistent low blood pressure that limited standing bouts duration. The described adjustments in stimulation parameters progressively improved functional outcomes represented by the transition from no independent lower limb extension, to bouts of independent extension of the left lower limb, to bouts of bilateral lower limb extension (FIG. 4D-E). This was associated with changes in activation pattern characteristics (FIG. 4F) resulting in trends of higher EMG amplitude, lower median frequency (MDF) and lower MDF standard deviation (for the right lower limb). As previously described by our group, these trends are consistent with an improved effectiveness of EMG pattern for standing with scES.

Subhead 5: Selection of Electrode Configuration for Facilitating Stepping

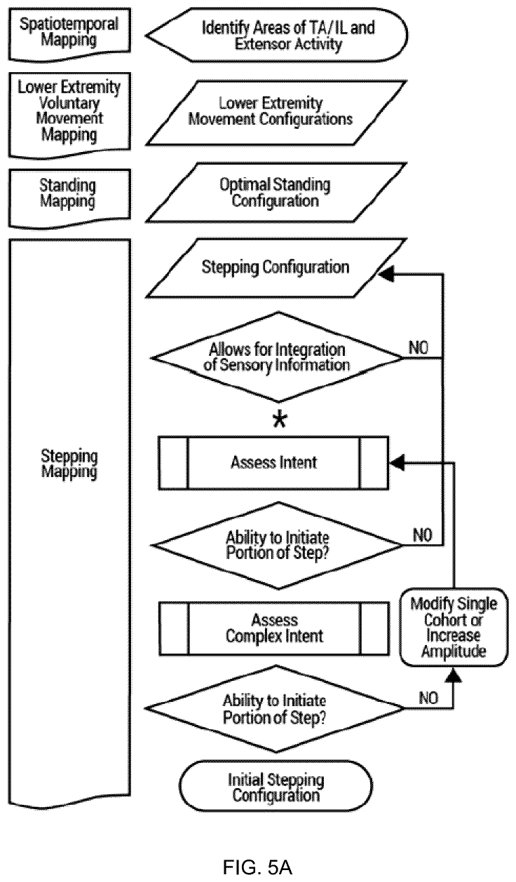

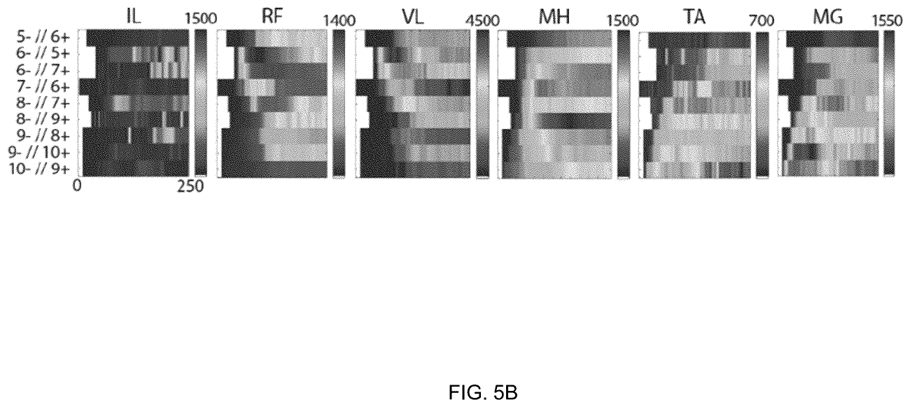

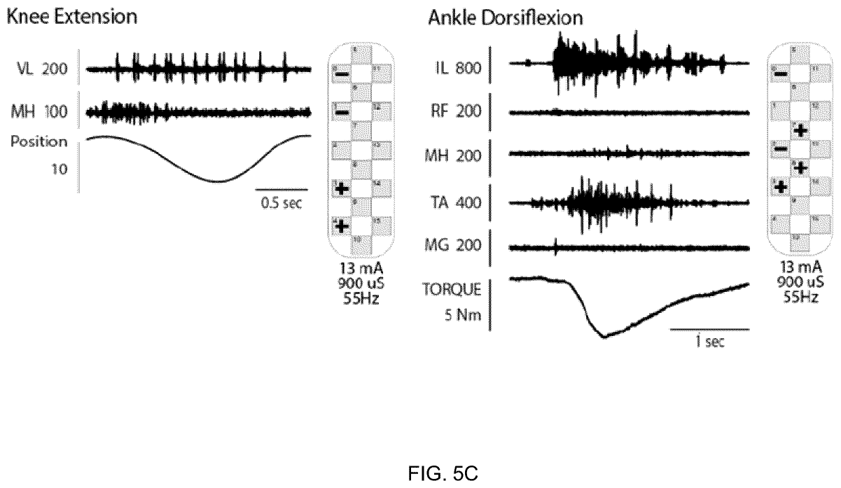

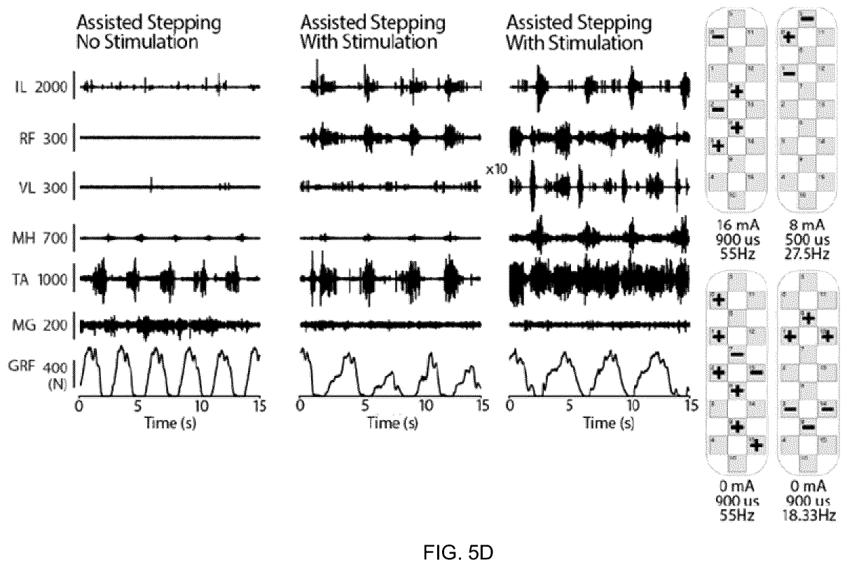

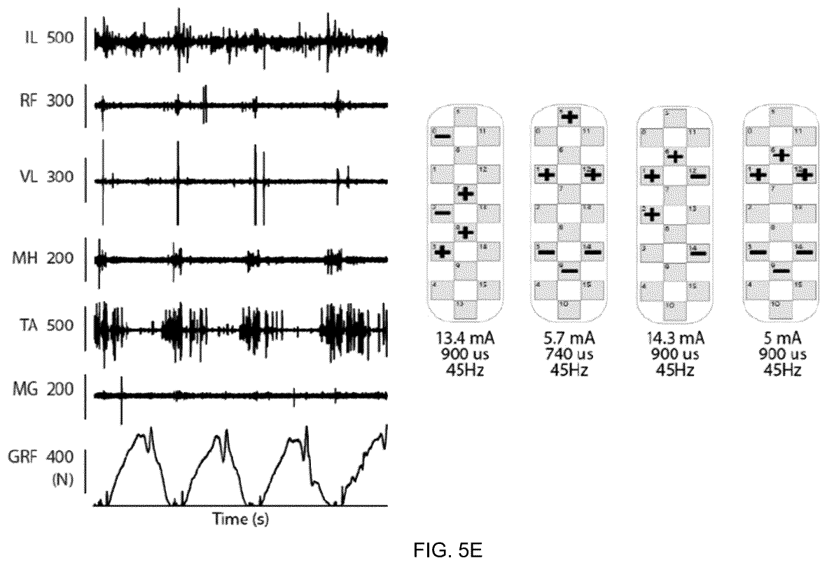

The selection of anode and cathode distribution, number of cohorts and stimulation frequencies are based on the spatio-temporal mapping data, in addition, the standing and voluntary configuration provide key information used in optimization (FIGS. 5A-C). The voluntary movement configuration informs the team on the location and distribution of electrodes for hip flexion and ankle dorsiflexion, key aspects of the swing phase. The standing configuration informs the team on areas that promote knee extension as well as frequencies required.

Although stepping is not a simple combination of functional tasks such as knee extension and hip flexion, the prior configurations allow for an initial starting point for the selection of cohorts. A cohort is defined as a group of active electrodes with a given amplitude, frequency and pulse width. Multiple cohorts are interleaved, in a tonic fashion, targeting a specific area of the spinal cord to enhance excitability promoting supraspinal signals and sensory information integration. In most cases, individuals will have left and right differences requiring a left and right ‘swing phase’ cohort. This cohort will either be focused on ankle dorsiflexion or hip flexion or a combination of both. Individuals having difficulty at any point of the swing phase might require multiple cohorts focused on this capacity. In addition, given the differences in electrode distribution and frequency required for standing, a single or multiple cohorts focused on extension are required during the early phase of mapping.

Mapping is initially performed on the treadmill with body weight support. Initial speeds and body weight support are found without stimulation (FIG. 5D). The excitability and coordination patterns observed without stimulation, with and without intent, will also inform the team as the modulatory potential of spinal circuitry in the presence of appropriate sensory information. Once stimulation is turned on, the team first observes the integration of scES and sensory information and how motor output in modulated. If stimulation continues to allow for integration to occurs and the motor output is appropriately modulated to the step cycle, the next step is to add intent (FIG. 5D). If stimulation generated an undesirable pattern such as excessive flexion or excessive extension that cannot be appropriately modulated with sensory information the stimulation parameters need to be changed.

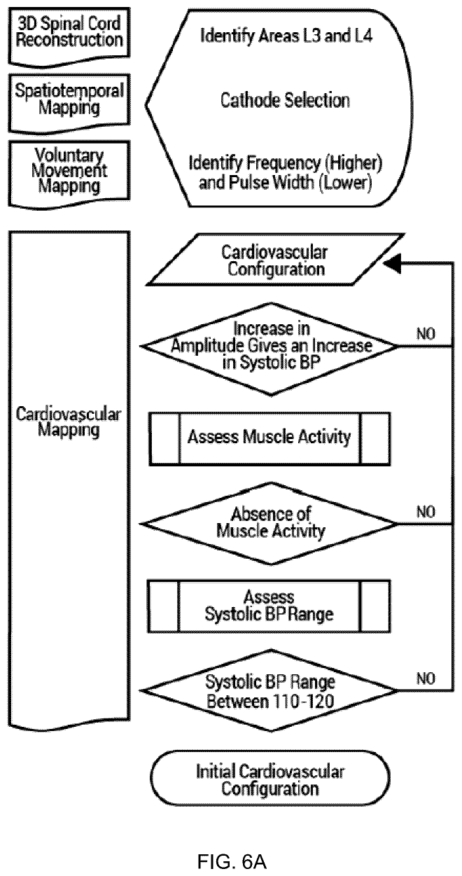

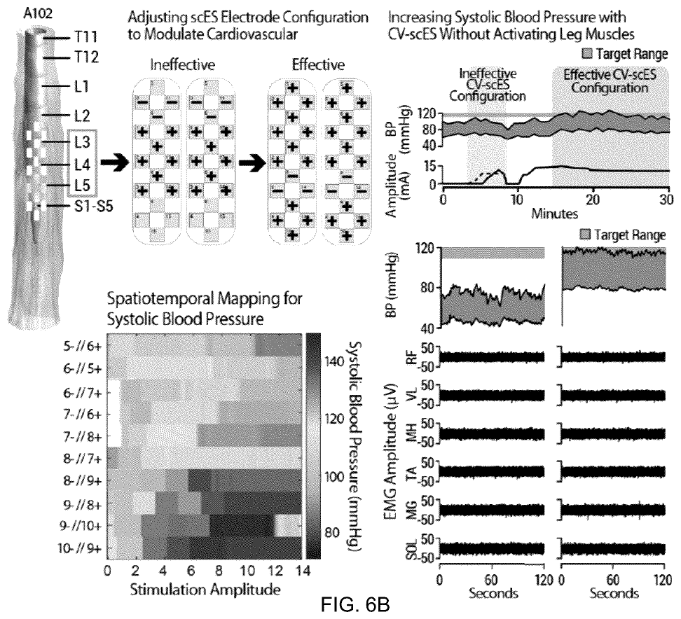

Once intent is integrated into the mapping process individuals will be asked to focus on a single component of the step cycle, i.e. left leg flexion during swing. The team will observe how intent modulates the EMG for the specific side, as well as the contralateral side. If modulation occurs on the appropriate agonist muscles, but no independence is observed, the stimulation intensity can be increased on the desired cohort. Stimulation intensity can be increased until independence is achieved (FIG. 6E) or stimulation becomes too strong that modulation is no longer appropriate. Care must be taken to make sure intent and/or an increase in stimulation does not cause the contralateral leg to also flex during the attempt.

Strategies that can be used in the optimization are slowing down the speed of the treadmill to allow the individuals to have more time to coordinate the intent to phase of the step cycle. Providing a general cohort, typically not side specific, that increases overall excitability of the network. This cohort becomes the key integrator for all additional cohorts and typically remains intact through future mapping adjustments. In most cases, a single leg will emerge as the ‘easier’ leg and that pattern will become automatic. A slow integration of other components and adjustment of configuration is typically required for coordinated stepping to emerge.

Subhead 6: Selection of Electrode Configuration for Cardiovascular Regulation

Referring now to FIGS. 6A and 6B, we first select the cathode distribution by identifying the location of L3-L4 spinal cord levels based on the Neuroanatomical 3D reconstruction model of spinal cord. The spatio-temporal mapping data identifies those cathodes that may have modulated blood pressure without eliciting EMG activity. The voluntary movement configuration provides information related to the frequencies and pulse widths optimized for motor activity. We select frequencies 20-30 Hz greater and pulse widths 2-3 times lower than those optimized for motor activity. We first select anodes directly adjacent to the selected cathodes and add additional anodes if muscle activity occurs, selecting them based on the cathodes effective for motor activity. Then we assess whether increasing amplitude directly increases systolic blood pressure and if so we increase the amplitude until reaching the target range (e.g., 110-120 mmHg systolic blood pressure). When a targeted upper limit is reached or exceeded (e.g., above 120 mmHg systolic blood pressure) we decrease the amplitude. We then assess for muscle activity adding anodes and/or increasing frequency if there are significant motor responses. Additional cohorts may be added to optimize the sensitivity of the blood pressure response and/or avoid muscle activity.

Subhead 7: Selection of Electrode Configuration for Facilitating Bladder Storage and Emptying

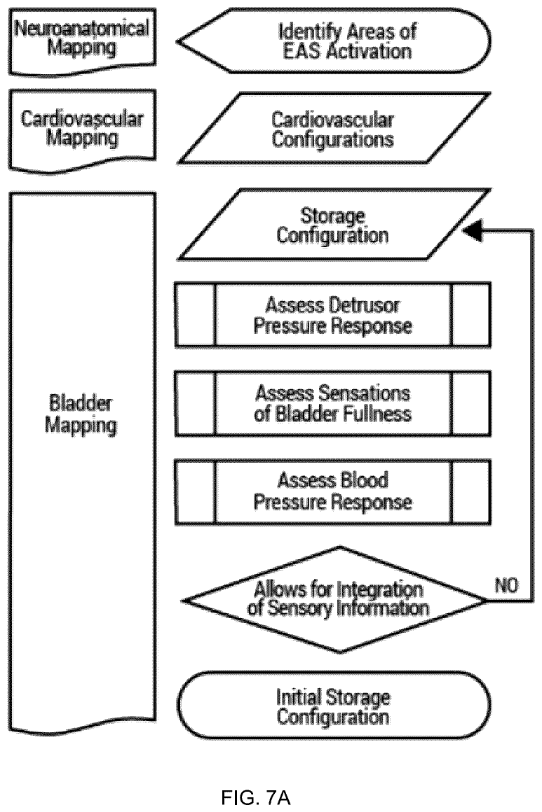

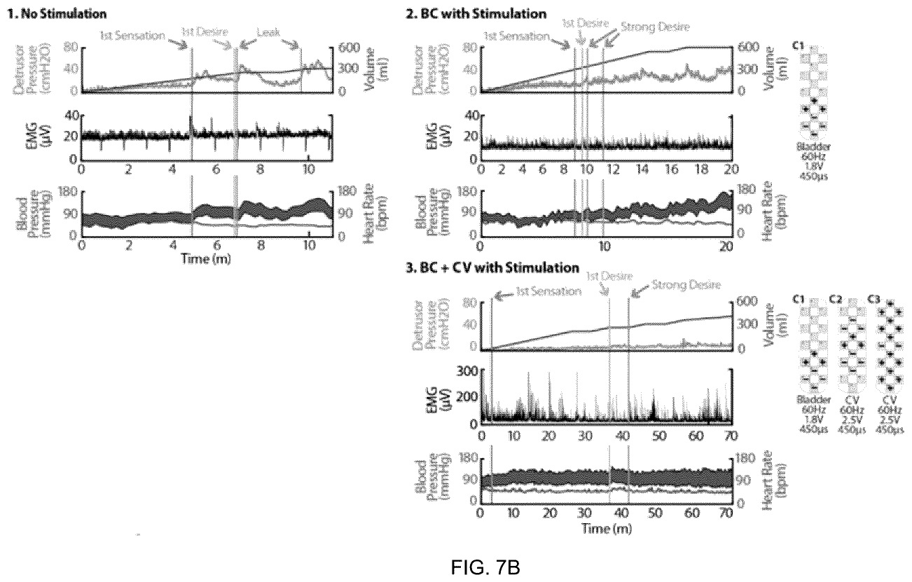

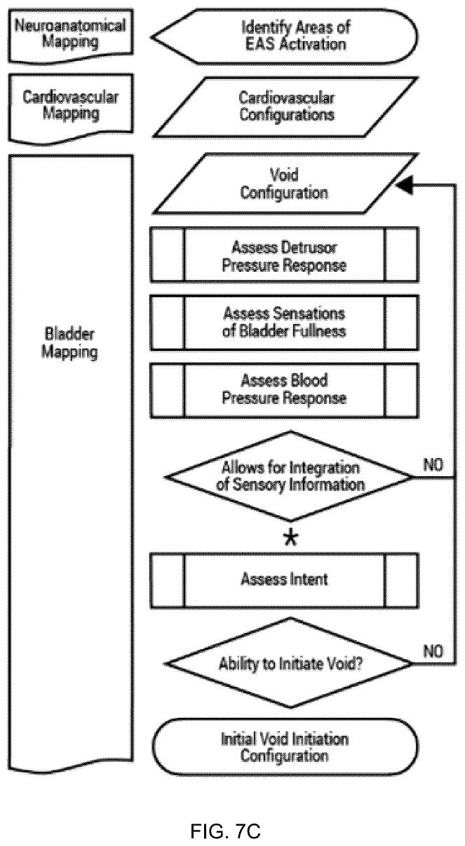

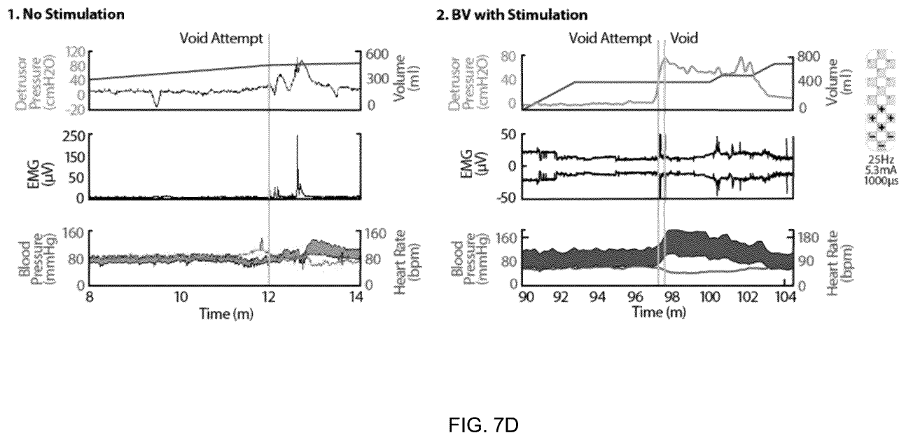

Referring now to FIGS. 7A-D, bladder mapping follows a human-guided interactive optimization approach where the experimental mapping process is subdivided into separate domains/tasks to isolate parameters for storage function and the initiation of voiding. Since these domains are inter-dependent, subsequent optimization is used to test and refine parameters concurrently in order to build a comprehensive framework for multi-system stimulation. Mapping sessions for each participant use the detrusor and urethral pressure responses as well as sphincter EMG responses during both storage and emptying phases (void attempts) to identify the scES parameters (anode, cathode selection; frequency and amplitude, and number of cohorts). The goal for bladder capacity BC-scES is to target volumes between 400-500 mL based on average normal capacity and avoiding over-distention in individuals performing intermittent catheterization 4-6 times/day (including average fluid intake). We also target filling pressures (<10 cmH₂O) to improve overall bladder compliance and detrusor leak-point pressures (<40 cmH₂O).

Based on previously published methods, bladder mapping is performed by selecting electrode configurations with cathodes in three different locations. Cathodes positioned caudally, target the sacral micturition center and parasympathetic pathways. Cathodes positioned in the mid-array target the purported lumbar spinal coordinating center with presynaptic connections to sphincter motoneurons. Finally, cathodes positioned rostrally, target sympathetic pathways (location selection order varied for each mapping session). Changes in detrusor pressure, sphincter activation/relaxation, and blood pressure responses are monitored while conducting a gradual ramp up of stimulation frequency and intensity until a near-motor threshold stimulation amplitude that does not elicit direct lower limb movements is selected. Stimulation frequency and intensity are then modulated synergistically in order to isolate an optimal frequency that elicits an overall continuous low detrusor pressure filling profile with a synchronized sphincter EMG pattern effective for bladder continence. Guided by participants' sensations of bladder fullness, the transition from continence to micturition aims to integrate ascending inputs and descending volitional drive. Electrodes in the caudal, middle, and rostral regions of the array are selected while frequency is kept constant and amplitude is adjusted in order to isolate an optimal intensity that drives the initiation of voiding activity (simultaneous increase in detrusor pressure with decrease in upper urethral pressure and quiescence of sphincter EMG responses). Electrode location and selection refinement is further modified to adjust for sensory and autonomic symptoms during mapping.

Lower extremity and trunk EMG is monitored continuously throughout mapping to identify those parameters that modulate detrusor pressure and coordination with the external anal sphincter muscle (mirroring external urethral sphincter), but do not elicit motor activity in the lower extremity or trunk. Stimulation amplitude is lowered, frequency increased, and electrode selection is modified to avoid lower extremity/trunk activity. All mapping urodynamic studies should be completed at least two days apart.

The motor and autonomic functional mapping methodology for tonic-interleaved scES and scTS provides clear strategies for simultaneous recovery of the myriad of consequences of severe, chronic spinal cord injury. Optimization of surgical or external placement of the electrode array is guided by anatomical markers using Neuroanatomical 3D reconstruction model of the spinal cord and intraopertive electrophysiology. Additionally, we highlight the importance of spatio-temporal mapping prior to starting functional and physiological mapping. Spatio-temporal mapping provides a clear relationship between electrode location and motor activation. Given the individuality of injuries and neurophysiological profiles following a spinal cord injury, mapping becomes a clear strategy for understanding spinal networks excitability levels and responses to stimulation. Functional stimulation configurations are complex in nature, needing the integration of knowledge acquired during spatio-temporal mapping about stimulation thresholds, motor pool relationships and frequency dependency. During functional mapping small changes in electrode selection (polarity and location) can have various effects on motor activity and blood pressure regulation, understanding how these parameters affect relationships between agonistic and antagonistic muscles helps reduce the iterations needed to optimize stimulation.

Although various strategies for stimulation and mapping have been employed, understanding of electrophysiological and functional responses are key to identify the initial successful configurations for training. EES uses computational simulations and imaging technology to identify motor hotspots and recruitment relationships relative to dorsal root anatomy. These strategies are used to reduce the mapping iterations assessed on each individual. Similarly, computational simulations use electrode position relative to anatomical structures to derive potential stimulation outputs. For EES to be effective it requires the delivery of recurring sequences of stimulation targeting specific motor hot spots at supra-threshold amplitudes. During training, wireless communication enables real-time control of stimulation parameters where sequences of stimulation are preprogrammed in either open loop or triggered closed loop (by external sensors/signals). This strategy supersedes the afferent feedback and the autonomous interneuronal circuitry. In contrast, scES allows for restoration of all essential functions of the spinal cord by receiving and integrating a variety of signals.

We have provided evidence that task specific neuromodulation is governed by the stimulation parameters selected and how activity based recovery training integrates to result in motor recovery in those with chronic spinal cord injury. The state of the network excitability is a key parameter in executing motor tasks. By finding the relationship between electrode selection, frequency and amplitude and the excitability state of the network, the researcher or clinician can control the modulation of the spinal circuitry and promote the integration of peripheral and supraspinal signals. This approach results in a muscle-specific feedback on the effectiveness of scES-promoted muscle activation for standing. This tactic can be implemented to identify which of the tested sets of stimulation parameters promote muscle activation more effective for standing even if the same level of external assistance is required. Additionally, it can contribute to detect which muscle groups are not activated properly, so that appropriate adjustments in stimulation parameters can be explored.

Integration of systems has to be considered during the functional mapping process. It is not uncommon to encounter an individual who requires assistance with blood pressure regulation during standing (FIG. 4E). In this case, knowledge of the optimized cardiovascular configuration will provide a starting point for integration with standing. Further, understanding the components of the specific configurations that suppress muscle activity are critical in the integration process. Similarly, bladder storage configurations will most likely require a configuration to lower blood pressure. Multi-system integration for scES and scTS is achieved by interleaving configurations focused on two or more functions and it is possible due to the single optimized placement of the electrode array. Physiological control and regulation typically handled by various systems prior to injury can be integrated through optimized scES or scTS neuromodulation to allow individuals with spinal cord injury to restore complex functions in the presence of stimulation.

Translation into clinical practice has to follow some components of the approach to mapping presented herein. The omission of spatio-temporal and functional mapping can lead to low levels of success and a potential disconnect between research outcomes and clinical outcomes. Although, thought to be time consuming and requiring electrophysiological knowledge the benefit associated with early optimization of configurations cannot be overstated. The institution of widely available databases with successful configurations identified with associated information and training modules would accelerate the translation to use in the clinic. Introduction of artificial intelligence and learning algorithms can reduce the burden by optimizing the mapping procedures and proposing initial configurations. This will not only reduce the time needed for the mapping sessions but potentially recommend functional configurations that provide an effective starting point for assessment.

Locomotion

An exemplary system and method for improvement of locomotion function in an individual with a spinal cord injury via electrical stimulation of the spinal cord with anatomical, electrical, and physiological specificity is provided here. This exemplary system and method serves a proof of the efficacy of spinal cord stimulation after implantation, mapping, and selection of electrodes as described above.

Prior research has shown partial recovery of locomotion through locomotor training, pharmaceutical agents, and electrical stimulation of the brain, spinal cord, and peripheral nerves. This is likely through neuromodulatory effects of these approaches enhancing neuroplasticity, reawakening dormant networks and/or creating new connections within and between spinal networks. Disclosed herein is a merging of these approaches to engage activity-dependent mechanisms that will generate a synergistic effect that can facilitate improved recruitment and coordination of motor pools leading toward locomotor recovery.

The instant systems and methods use a combinatorial approach of non-invasive multi-site spinal cord stimulation with use-dependent non-weight-bearing training (nWBT) and weight-bearing training (WBT) to enhance neuroplasticity of the spinal pathways and networks to facilitate stepping behaviors on a 27-year-old participant with a spinal cord injury at T2 classified as AIS level A. In-silico simulations in a virtual phantom were performed to assess the specific neural structures targeted by each individual stimulation site. Behavioral assessments were carried out to evaluate his ability to move his legs without weight-bearing in a Gravity Neutral Device (GND) and during weight-bearing on a Body-Weight Support Treadmill (BWS-TM) to assess activation of his leg muscles through electromyography (EMG) and kinematics. Neurophysiological assessments evaluated the state of supra- and sublesional connectivity through multiple pathways and systems by using a paradigm of supralesional conditioning of motor evoked potentials (MEPs). MEPs were triggered through spinal stimulation at the lumbar spinal cord after conditioning stimuli were delivered above the site of injury. Motor responses of the leg muscles were collected and analyzed (ref). Overall, these assessments showed that locomotor function improved after training with scTS likely through changes in the spinal pathways and networks. These results suggest that rehabilitative strategies, when combined with scTS, can facilitate recovery of function after SCI.

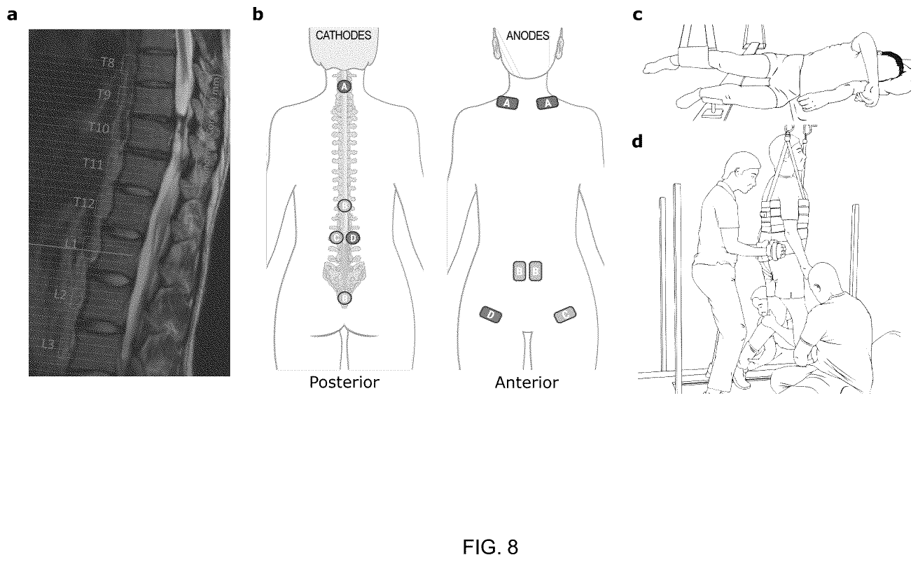

The participant is a 28-year-old Caucasian male that presented with a motor complete paraplegia after a discomplete SCI at T8-T11. Extent of the injury was verified through magnetic resonance imaging (FIG. 8a). The participant was classified as AIS-A with a neurological level of injury at T1 with a partial motor and sensorial preservation zone up to T7 and T9, respectively, as determined by an International Standards for Neurological Classification of SCI (ISNCSCI) examination. Damage to the spinal cord was due to indirect trauma lateral to the spinal cord 5 years prior to study enrollment. No supportive vertebral hardware was implanted. The participant reported regular use of cannabis for pain control and recreational purposes. There was no reported intake of other pharmacological forms of pain control. The participant did participate in a study assessing the acute effects of 5-HTP during the first week of training. However, there is no evidence to support that this had a significant effect after a month or 3 months of training.

Previous studies have shown that standing and step-like behaviors can be elicited by stimulation at the L1/L2 and T11/T12 vertebral levels, respectively. However, early mapping sessions during non-weight bearing in the GND showed that a more caudal site, L2/L3, with anodes placed along the inguinal ligament provided a more consistent and effective knee extension than the L1/L2 site suggested by previous studies. Additionally, to provide function-specific stimulation, electrodes were placed bilaterally for left-right specificity. Stimulation was delivered anti-phase with a fixed pulse-train duration in nWBT and in a closed-loop manner in WBT where gyroscopes attached above the knee triggered stimulation during the stance phases of the gait cycle for each leg. Other studies have also shown that scTS of the cervical area can lead to facilitation of voluntary behaviors. Lastly, stimulation of the pudendal nerve has been shown to effective neuromodulator of the lower limb muscles. The instant systems and methods replicate this effect through scTS at the sacral-coccygeal level.

Referring now to FIG. 8, scTS was delivered at five different locations, midline C3/C4, midline T11/T12, bilaterally at L2/L3, and midline on the sacral-coccygeal region to comprehensively neuromodulate the entire locomotor network. Circular cathodes (1.25′ PALS®, Axelgaard, Fallbrook, CA) were placed in the aforementioned locations, while anodes (2′×3.5′ PALS®, Axelgaard, Fallbrook, CA) were placed on the clavicles for the cervical cathodes, on the rectus abdominis for the T11/T12 and sacral-coccygeal cathodes, and ipsilaterally along the inguinal ligament for the respective L2/L3 cathodes. Placement of the cathodes and anodes can be seen in FIG. 8, panel B. scTS comprised biphasic pulses at the C3/C4 and T11/T12 locations, and of monophasic pulses at the other locations. Pulse width duration was 1 msec for all sites and stimulation frequency was set to 30 Hz for all midline sites and at 40 Hz for the L1/L2 sites. These parameters were determined through multiple mapping sessions prior to training. A modulating carrier frequency at 5 kHz was used to minimize sensory sensitivity to scTS. Current amplitude was determined at the start of each session and set to be at motor threshold unless the participant reported discomfort from the stimulation. Motor threshold was determined by EMG, visual observation of muscle contraction and/or movement.

The participant underwent locomotor training with scTS five times a week for three months. Locomotor training was divided into two phases: an initial phase of one month in which the participant received scTS with nWBT in a GND carrying out voluntary tasks and passive locomotion through trainer assistance, and a second phase lasting two months in which scTS was delivered during WBT on a BWS-TM (ref). When in the GND (FIG. 8, Panel C), the participant was lying recumbent with their legs suspended to reduce the effort required to move the lower limbs against gravity. Here, the participant was asked to perform two tasks: to attempt voluntary hip and knee extension for each leg and to swing their legs in a rhythmic step-like manner, adding arm swings during their attempt. During WBT in the BWS-TM (FIG. 8, Panel D), the participant is asked to attempt stepping while trainers are positioning and assisting hip, leg, and ankles. These sessions are further subdivided into bouts at a fast treadmill speed (1.5-2.0 m/s) to elevate the overall excitability of the locomotor network and thus train the automaticity of the locomotor CPG, and at slow speeds (0.5 m/s) to drive voluntary effort and thus strengthen corticospinal pathways. In both nWBT and WBT, scTS was delivered concurrently with the training.

Kinematic data was collected and analyzed using a 5-camera motion capture system with Cortex software (Motion Analysis Co., Rohnert Park, CA). In the GND, the participant lies recumbent on their left side and reflective markers are placed at the right shoulder, right hip, right knee, right ankle and right toe to obtain hip, knee, and ankle angles of the right leg. Kinematic data was collected at a sampling rate of 100 Hz. The participant was asked to attempt to move his legs in a step-like manner, focusing on evoking and maintaining this behavior. Kinematic data was collected at baseline, after GND training, and after BWS-TM training.

Electromyography (EMG) of the lower limbs was collected while weight-bearing stepping in the BWS-TM to assess changes in muscle activation after BWS-TM training. EMG of the rectus femoris (RF), vastus lateralis (VL), medial hamstring (MH), tibialis anterior (TA), medial gastrocnemius (MG), and soleus (SOL) muscles was collected using surface EMG electrodes (MA400, Motion Labs Systems, Baton Rouge, LA) at a sampling rate of 2,000 Hz. EMG was then filtered using an adaptive filtering technique to remove background noise. EMG was analyzed to obtain total power and mean frequency. Muscle coordination between agonist and antagonist pairs was assessed by calculating the relative Pearson's correlation coefficient of the envelope of the normalized EMG between muscle pairs, where the correlation coefficient of antagonist muscles is inverted, such that a value of 1 dictates appropriate antagonistic activation. A positive change in the relative Pearson's correlation coefficient dictated improvement in muscle coordination while a negative change indicated decline.

Modulation of MEPs at the lumbar region through conditioning stimuli has been used in several studies to evaluate the patency of descending tracts through a damaged spinal cord. Here, we utilized these approaches to assess MEP conditioning through cervico-lumbar pathways (CLPs), the propriospinal system (PPS), and reticulospinal pathways (RSP). Additionally, a novel paradigm to evaluate the state of the conditioning stimulus. To condition through the RSP, an acoustic startle reflex was elicited using a 700 Hz tone at 80 dB. To condition the locomotor network through CSP, the participant was asked to perform a task at the onset of an auditory cue for a duration of 3 sec. The tasks were left and right plantarflexion, left and right dorsiflexion, and bilateral knee extension and flexion. corticospinal pathways (CSP) using a task-driven paradigm was also developed.

A conditioning stimulus was delivered at a site above injury that pertains to each pathway. To condition through the PSS, a stimulus was delivered at the ulnar nerve of the right arm. For the CLPs, a single pulse delivered through a scTS electrode located midline at the C3/C4 vertebral level served as the conditioning stimulus. To condition through the RSP, an acoustic startle reflex was elicited using a 700 Hz tone at 80 dB. To condition the locomotor network through CSP, the participant was asked to perform a task at the onset of an auditory cue for a duration of 3 sec. The tasks were left and right plantarflexion, left and right dorsiflexion, and bilateral knee extension and flexion.

To elicit a MEP in each of the conditioning modalities, a secondary stimulus was delivered at a site below the injury at L1/L2 vertebral level 90 msec after the stimuli for the CLPs, PPS, and RSP. For CSP conditioning, a stimulus was delivered at a site below the injury at L1/L2 during the voluntary task (1000 msec after tone onset). The amplitude of the second stimuli was selected such that activation of all muscles was observed but was not at maximum or minimum of any muscle to ensure that both inhibition and facilitation of MEPs can occur.

A minimum of three conditioned responses, along with control MEPs, were collected at each time point. A difference in the area under the curve (AUC) of the MEP with respect to the control stimulus indicated modulation through each respective system or pathway. Muscle responses were collected through Spike2 software (CED Ltd, Cambridge, England) at a sampling rate of 5000 Hz. MEPs were collected from the RF, VL, MH, TA, MG, and SOL muscles. The data was processed and analyzed through MATLAB 2020A (Mathworks Inc, Natick, MA). Assessment of the CSP was performed only after nWBT and after WBT.

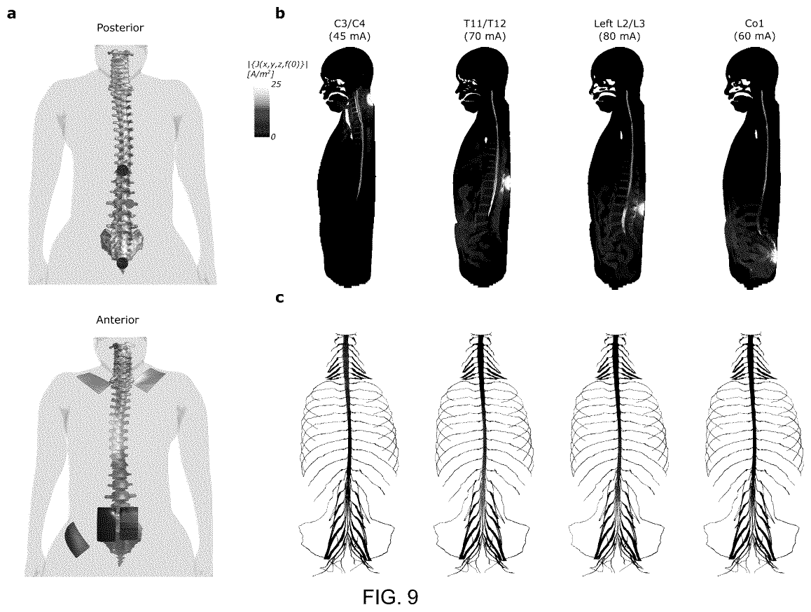

Referring now to FIG. 9, current field distribution simulations were performed to elucidate the neural structures targeted by scTS at each of the stimulation sites (FIG. 9, Panel A). Ohmic quasi-static simulations were performed using a finite element model approach (Sim4Life, Zurich Med Tech, Switzerland) in a virtual model (Yoon Sun ViP 4.0, IT'IS Foundation) with tissue-specific electrical properties. Current amplitudes used in the simulation matched average amplitudes used experimentally. The current field density was used to assess the foci of stimulation with simultaneous scTS.

Computational modeling of the current distribution throughout the virtual phantom shows that, while relatively widespread, scTS at the different sites is able to stimulate distinct neural structures (FIG. 9, Panels B and C). The C3/C4 cathode increases current density around the C5 spinal segment but is distinctively concentrated along the C5 and C6 dorsal roots. Stimulation between the T11/T12 vertebrae causes a spike in concentration along L2 spinal segment, but also likely stimulates all the spinal roots below the L2 segment. Similarly, the L2/L3 lateral cathode stimulates a vast majority of the lumbar roots, however, only below the L4 level. Finally, stimulation at the Col leads to stimulation of only the S3-S4 roots.

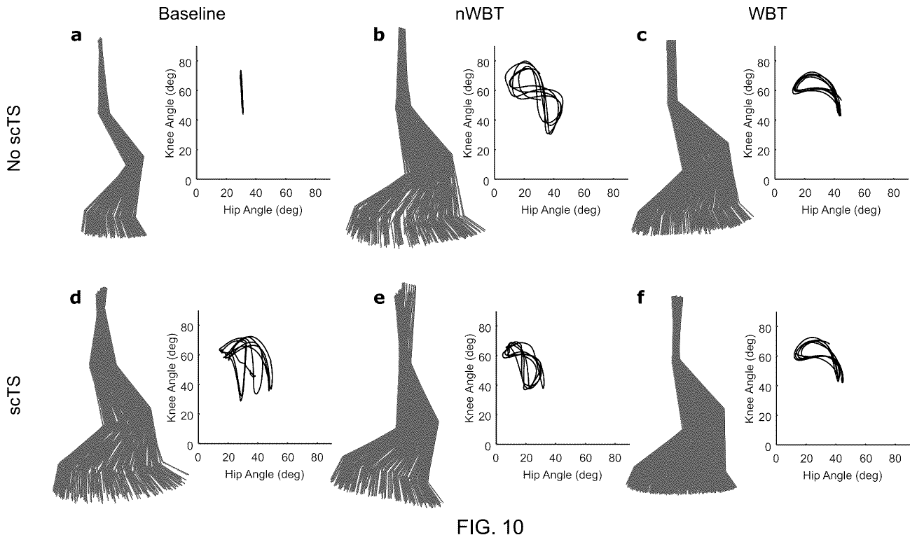

Referring now to FIG. 10, baseline testing of hip and knee kinematics in a gravity neutral setting showed very limited movement, with only a 1.9° range in hip angle and a 29.5° range in knee angle (FIG. 10, Panel A). After GND training (FIG. 10, Panel B), however, these angles ranges increased to 38.7° and 19.4°, respectively. After TM training (FIG. 10, Panel C), there was a reduction in the total knee angle to 32.8° at the hip and 29.6° at the knee, however, the kinematic pattern follows a more coordinated stepping pattern that more closely resembles normal knee-hip dynamics (ref). Ankle angles saw no considerable change and were less than 5° at all time points. With the addition of scTS during baseline (FIG. 10, Panel D), hip angles increased dramatically, however coordination between the hip and knee was not optimal and was highly variable. After nWBT (FIG. 10, Panel E), while the effect of scTS with training was positive, once stimulation was applied joint angles decreased compared to when no scTS was delivered. Finally, after BWS-TM training (FIG. 10, Panel F), there was no evident change in joint angles and the appropriate pattern observed without scTS remained.

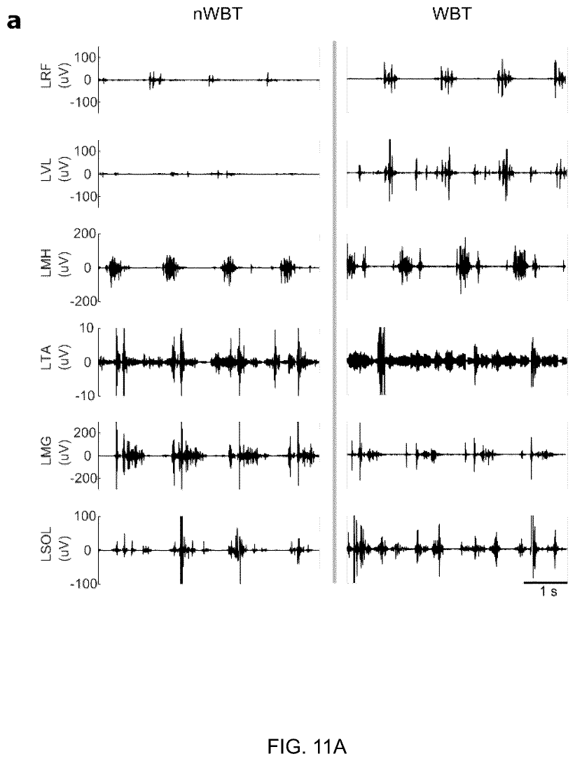

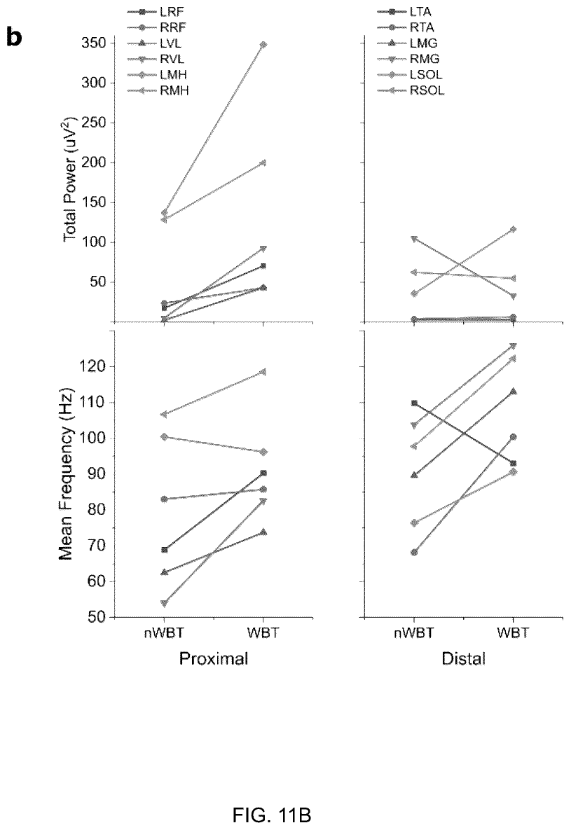

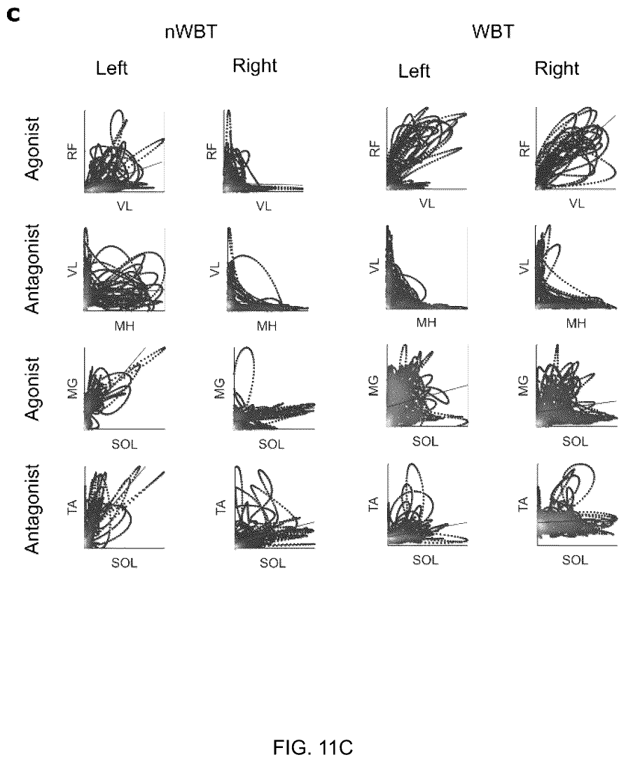

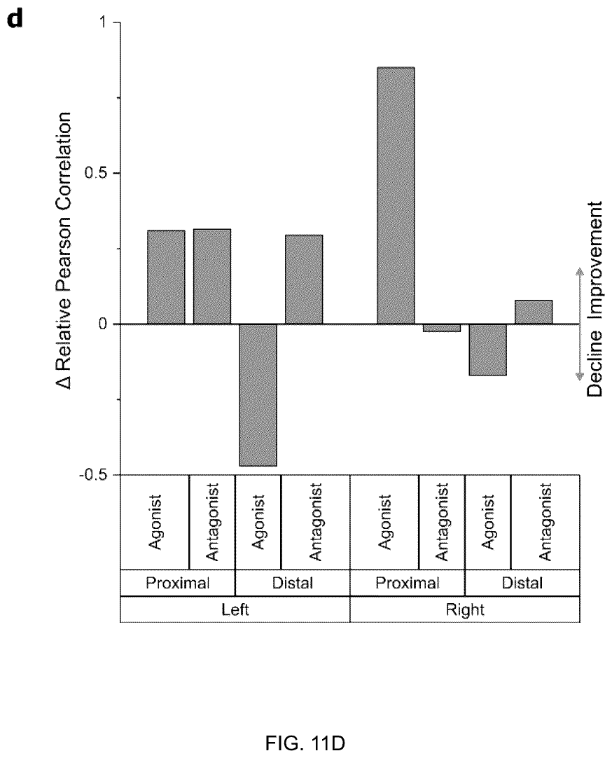

Assessments on the BWS-TM showed changes in EMG power and mean frequency, as well as changes in muscle activation patterns between muscle sets. FIG. 11A shows EMG traces of the left leg muscles before WBT and after training. Analysis of EMG properties (FIG. 11B) shows that WBT led to an increase in total power in the proximal muscles. The distal muscles, however, did not show a significant difference in power. Both proximal and distal muscles showed significant increase in the mean frequency. Looking at the correlation between agonist and antagonist pairs shows that after training (FIG. 11C), there is an overall increase in coordination, with the relative correlation increasing among all muscle pairs except for the distal agonists (FIG. 11D). When assessing coordination between muscles of both legs, an average increase of 0.58 was observed in the change in relative Pearson's correlation coefficient was observed for bilateral agonist proximal muscles, and an average decrease of 0.14 observed in antagonistic muscles. Distal muscles did not show an improvement when compared to before WBT.

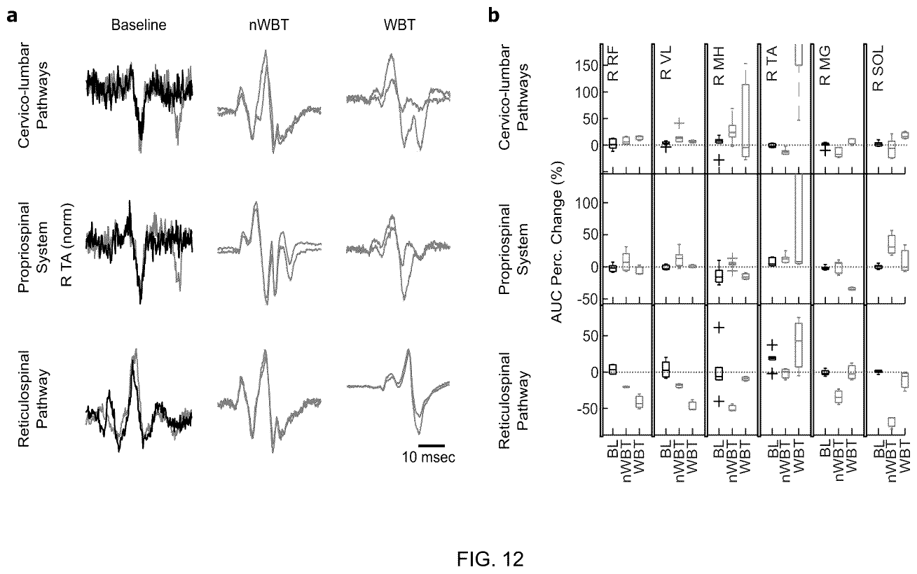

Neurophysiological studies assessing the conditioning of sublesional locomotor MEPs suggest that training with scTS, without or with weight-bearing, can lead to re-emergence of supralesional modulation of the locomotor network differentially through the multiple spinal pathways. FIG. 12, Panel A shows average non-conditioned and conditioned MEP responses of the right TA muscle for each conditioning paradigm. FIG. 12, Panel B shows that the percent change in AUC with respect to non-conditioned MEPs changes differentially between muscles, across different pathways tested, and after nWBT and WBT. The largest overall change in MEP responses were observed when conditioning MEPs through the RSP. Albeit, contrary to expected, the changes in MEP conditioning through the RSP are mostly of an inhibitory nature. However, since the non-specific CLP modulation did cause a mostly facilitatory change, it is possible that the inhibitory response observed in RSP modulation is due to changes at the supraspinal level, rather than spinal neuroplasticity.

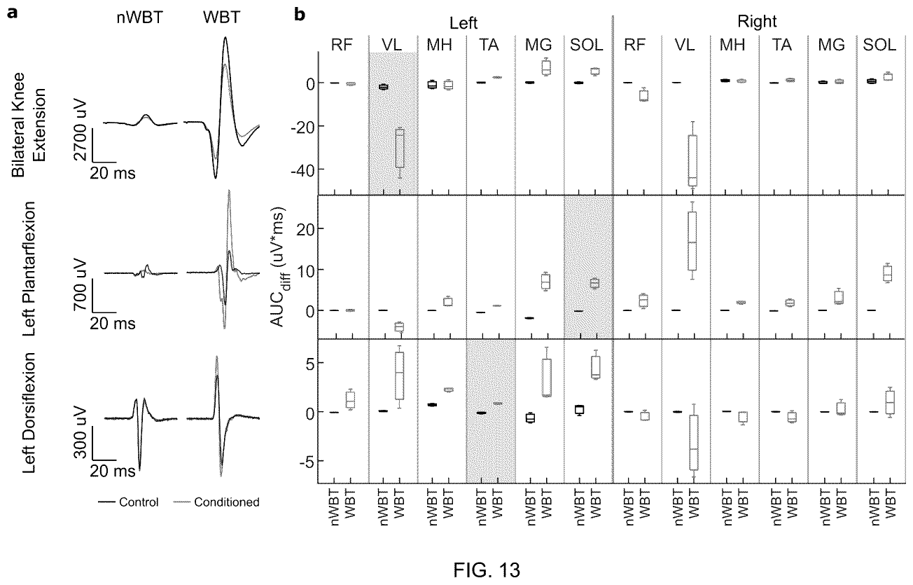

Corticospinal conditioning through the voluntary intent tasks showed that after nWBT, modulation of MEPs was minimal or not present in the main muscles involved during these tasks. After WBT, there is evidence of modulation of the MEPs in all main muscles (FIG. 13, Panel A). Paradoxically, while still showing modulation, left VL shows inhibition rather than facilitation of the MEPs during knee extension. FIG. 13, Panel B shows the AUC when compared to control pulses at each timepoint across all muscles. There is observable task-specific modulation across all muscles, with bilateral knee extension heavily modulating bilateral VL, left plantarflexion modulating bilateral MG and SOL, and left dorsiflexion facilitating left VL, MG, and SOL, but inhibiting right VL.

Transcutaneous stimulation of the spinal cord has shown in multiple studies to neuromodulate posture and locomotion (refs). Furthermore, animal studies have shown that scTS can also evoke plasticity at the spinal cord level, improving connections and reactivating dormant networks (refs). The results of this case study suggest that this neuroplastic effect can be enhanced by use-dependent nWBT and WBT, with each approach affecting locomotion differentially.

Computational simulations showed that the multi-site scTS configuration used in this study targets multiple sites along the spinal cord and various spinal roots. ScTS at the C3/C4 and T11/T12 levels showed a clear concentration of current along the surface of the spinal cord, however, sites L2/L3 and Col, showed high current density concentrations along the cauda equina, with Col stimulating only a subset of these spinal roots (S2-S4). While these results show that the T11/T12 and the lateral L2/L3 seem to target very similar structures with significant overlap, data collected during nWBT training showed very distinct behaviors when stimulating independently. Stimulation a the lateral L2/L3 sites allowed significant facilitation of knee extension, with noticeable lateralized specificity. This was not observed with stimulation at T11/T12. It is highly likely that these differences between the simulations and the experimental studies are due to how the spinal network, a much more complex system, behaves depending on which specific neural structure, or a combination of them, are excited by multi-site scTS. The current computational model used does not model spinal pathways nor interneuronal networks, and as such is unable to demonstrate any behavioral outcomes. However, this computational work remains of value, as it serves to distinguish which structures are likely to be stimulated by multi-site scTS; the first step in the cascade of events that lead to a functional change.

Kinematics showed that after non-weight bearing stepping, hip angles dramatically increased during voluntary stepping in the GND. However, the hip-knee cyclograms show a “figure 8” pattern that suggests that control of the knee joint is dependent on hip mechanics more so than through voluntary control of the joint. This is further reiterated through EMG recordings done in in a BWS-TM, where knee flexor activity overshadows that of knee extensors. Furthermore, EMG coordination analysis also showed low correlation between knee extensors, suggesting impaired proximal agonist muscle coordination, which may be preventing adequate knee extension. After BWS-TM training, this pattern disappears and a pattern showing a more active knee movement appears. This suggests more involvement of the knee joint which is now being actively controlled to resist the force of hip momentum. This final hip-knee angle pattern closely resembles a traditional hip-knee cyclogram seen in non-disabled people. EMG after BWS-TM Training collected in the BWS-TM confirms a comparatively more active knee joint muscles by showing a substantial increase in VL EMG power and improved correlation between right VL-RF activation. MEPs collected during voluntary tasks also showed clear VL modulation when compared to control MEPs. However, while this shows that corticospinal connections to the locomotor network are present, inhibition is observed rather than facilitation. A possible reason being changes in afferent input during these voluntary tasks when compared to what the setting is during BWS-TM Training, where weight-bearing leads to loading of the muscles. This difference in afferent input may be the cause of this inhibitory response, rather than excitatory response. It is also possible that the cortex has yet to completely associate a desired voluntary task to specific behaviors and thus selective activation of muscles is still not optimal.

Application of stimulation during non-weight bearing stepping also led to a different effect across different time points. It is possible that scTS played different roles across time points as a result of neuroplastic changes. Prior to training, it increased overall locomotor muscle activity. After nWBT, scTS may have shifted to improving control of the hip joint, reducing angles as a result. After WBT, since the afferent information during weight bearing has shown to play a crucial role in locomotion, scTS may instead serves as a supplementary mechanism that serves more to control fine movement, rather than facilitate gross movement.

Despite there was an overall improvement in hip and knee kinematics and proximal muscle activity after WBT, the same cannot be said for the ankle joint and distal muscles. After WBT, no change was observed in the ankle angles during GND assessments and while an increase in SOL power was observed, a large decrease in TA activation occurred. We surmise that this is due to the functional aspect of WBT, where focus is placed on training for standing and for support during stance. During these activities, plantarflexion is desired, i.e. maintaining SOL and MG activity constant to prevent forward bend of the ankle. Thus, scTS was mapped to facilitate plantarflexion during weight-bearing. Across 40 training sessions, this led to an overall decrease in dorsiflexor muscle activity and an increase in plantarflexor activity. This can be further shown when assessing facilitation of MEPs, where, during dorsiflexion, we observe much larger facilitation of MG and SOL and relatively smaller facilitation of TA.

While this was a limited case study of an ongoing larger study, neural changes occurring as a result of scTS and locomotor training were clear. While kinematics and EMG during stepping tasks provide information on the overall functional recovery of locomotion, it is with the neurophysiological studies assessing supraspinal conditioning of MEPs below the injury that a strong effect of neuroplasticity induced by the training on the spinal networks is seen. Changes were observed across all pathways tested, however, the RSP proved to have undergone the most change, while the PPS showed minimal changes. Of note were the changes in the CSP, where each task led to differential modulation across muscles, which was not evidently observed prior to WBT. This is evidence that new pathways coming from the cortex have been created or that dormant pathways across the lesion have been reactivated. This paradigm could also be used in future studies to assess the specificity by which cortical drive can facilitate specific muscle groups below the level of the injury, with specificity increasing as locomotor function is restored.

This case study provides evidence that locomotor training can be enhanced by the addition of scTS and this promotes neuroplasticity to the extent that activity is restored to previously quiescent muscles and modulation of the locomotor spinal network through corticospinal pathways is regained.

Bladder

An exemplary system and method for improvement of bladder function in an individual with a spinal cord injury via electrical stimulation of the spinal cord with anatomical, electrical, and physiological specificity is provided here.

Neurogenic bladder dysfunction is highly prevalent following a SCI, profoundly impacting health and quality of life. Loss of volitional control of micturition, consistent with an upper motor neuron-type injury, is accompanied by detrusor overactivity and detrusor-sphincter dyssynergia, where simultaneous detrusor and urinary sphincter contractions lead to high bladder pressure and insufficient emptying. Major urological concerns contributing to increased morbidity and mortality include incontinence, repeated lower urinary tract (LUT) infections that can result in sepsis, chronic vesicoureteral reflux, and hydronephrosis with progression to renal insufficiency. Furthermore, SCI above the sixth thoracic vertebra (T6) impairs cardiovascular reflexes, leading to autonomic dysreflexia (sudden elevation of blood pressure greater than 20 mm Hg above one's usual baseline) that limits bladder storage. Standard management of LUT dysfunction post-SCI includes a combination of pharmacological and catheterization approaches for storage and emptying, respectively, or insertion of an indwelling catheter when hand function is limited. While these measures preserve upper tract function, they do not address the potential to regain LUT control and further independence over time.

Restoration of bladder function is rated as a high priority among individuals with SCI. A recent survey investigating consumer needs and priorities indicates a strong desire and willingness to adopt neuromodulation interventions to facilitate a return to more normal bladder function and help reduce secondary complications negatively impacting quality of life. Lumbosacral spinal cord epidural stimulation (scES) combined with intensive activity-based recovery training is one such neuromodulatory approach that re-engages existing spinal circuits below the level of injury, challenging the novel post-injury circuitry to reorganize in functionally and physiologically significant ways. Capitalizing on the inherent functional capacity that comprises these systemic circuits, scES enables autonomic circuits to recover significant levels of function. We have previously shown that scES can be used to augment the lumbosacral neural circuitry below the level of injury sufficient to potentiate gains in bladder function achieved through activity-based recovery interventions alone. Driven in part by consumer demand as well as from a paradigm shift in rehabilitative strategies focusing on a return to pre-injury function, there is a critical need for a therapeutic intervention that aims to restore normal or even partial LUT function. The standard of care, which includes anticholinergic therapy and chronic catheterization, has high rates of discontinuation and an increased chance of diminishing bladder compliance with time (indwelling catheters), respectively. Both approaches require life-long maintenance and have adverse side effects leading to recurring illness and reduced quality of life. Importantly, cardiovascular complications associated with autonomic dysregulation post-SCI directly interfere with the ability to recover bladder function. Despite these improvements, much remains unknown about the potential of and how best to use scES to improve bladder function after SCI. Further development of the parameters of stimulation and programming strategies and protocols for improving bladder control and managing interactions from the cardiovascular system are also needed to advance neuromodulatory approaches.

In the current pilot trial, LUT function and blood pressure responses to bladder distention were examined throughout a targeted scES mapping study on an initial cohort of individuals (n=7). Appropriately selected stimulation parameters identified through scES bladder mapping were found to modulate local spinal reflexes important for both the maintenance of urinary continence and the initiation of voiding. Mapping of the overlapping 5-6-5 paddle array on each participant's reconstructed 3-D spinal cord was also performed to better understand the inherent anatomical variability of the vertebral column with respect to the lumbosacral enlargement and location of the conus tip across subjects. Finite element modeling was conducted to quantify the current density and distribution patterns generated by specific bladder cohorts, identifying the spinal cord locations optimal for modulating bladder storage and emptying. The selectivity and depth for targeting neural structures by scES as well as identifying the anatomical variability between individuals are critical to personalizing neuromodulatory strategies for paralysis.

The clinical and demographic information for enrolled research participants is provided in Table 6. Characteristics represented in the table were determined from the time at which each participant enrolled in the study. Participant ages ranged from 26-39 years of age (32.1±4.6), with a 6:1 ratio of males to females and an average time since injury at 9.1±2.5 years. All participants were assessed as motor complete SCI, with the level of injury ranging from C3-T2. Three participants managed their bladders with a suprapubic (SP) catheter and four participants performed clean intermittent catheterization (CIC).

TABLE 6
Participant Characteristics

| | | | | | Years Post | | Bladder |
|---|---|---|---|---|---|---|---|
| Participant | Age | | Neuro | AIS | Injury | Anal | Emptying |
| ID | (years) | Sex | Level | grade | (years) | Sensation | Method |
| A101 | 32 | M | C3 | A | 9 | No | SP |
| A96 | 27 | F | C4 | A | 5 | No | SP |
| B23 | 36 | M | C5 | B | 8 | Yes | SP |
| B21 | 33 | M | C4 | B | 11 | Yes | CIC |
| B24 | 26 | M | C7 | B | 8 | Yes | CIC |
| A68 | 39 | M | C8 | B | 10 | Yes | CIC |
| B07 | 32 | M | T2 | B | 13 | Yes | CIC |

AIS, American Spinal Injury Association Impairment Scale;
CIC, clean intermittent catheterization;
SP, suprapubic catheter

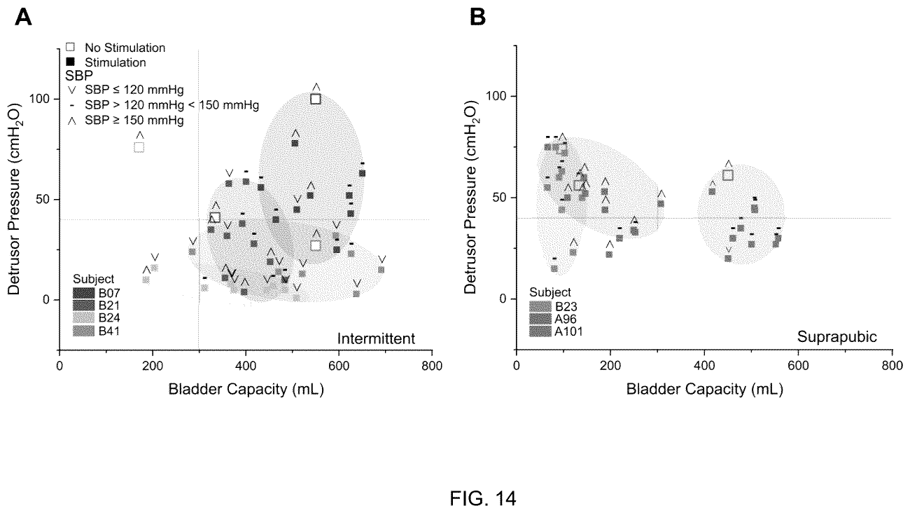

Referring now to FIG. 14, maximum cystometric capacity values and corresponding detrusor and systolic blood pressure values attained without scES (open squares) and during BC-scES mapping sessions (closed squares) are plotted for those performing CIC (FIG. 14, Panel A) and those using SP catheters (FIG. 14, Panel B). Pre-mapping bladder outcomes (open square plots) from urodynamic testing in the CIC group revealed average bladder capacity within normative ranges, per ICS guidelines (300-600 ml, optimal ranges in lower right quadrant). However, average detrusor pressure and systolic blood pressure values at maximum capacity were elevated above normative ranges (40 cmH₂O—detrusor pressure, upper quadrants; i.e. 110-120 mmHg-blood pressure). Bladder capacity values for the SP group were below normative storage values for ⅔ participants. Maximum detrusor pressure and systolic blood pressure values were elevated above normative ranges for all 3 participants. BC-scES mapping targeted parameters that promoted an increase in capacity while reducing maximum detrusor pressure and systolic blood pressure responses to bladder distention for both groups. The measurement trends for each participant are illustrated with the shaded ellipses.

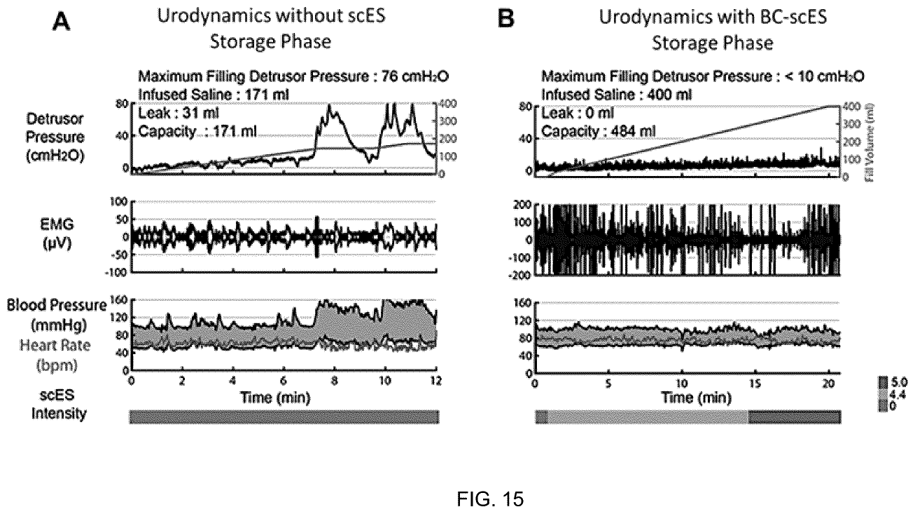

Referring now to FIG. 15, a representative example of the detrusor pressure-volume relationship, sphincter electromyography (EMG) responses, and interaction from the cardiovascular system without scES and with targeted BC-scES is provided in FIG. 15, Panels A and B, respectively. Without scES, detrusor responses to increased bladder volume exhibited instability marked by sharp and sustained increases in detrusor pressure or neurogenic detrusor overactivity (FIG. 9, Panel A). Additionally, detrusor pressure rose above clinically-recommended thresholds for bladder filling (>10 cm H₂O) and detrusor leak-point pressures (>40 cmH₂O). Furthermore, timed with each non-voiding contraction was an increase in systolic blood pressure, which remained elevated and outside the normative reference range (i.e. 110-120 mmHg), resulting in cessation of bladder filling, removal of residual volume, and a subsequent return to pre-fill arterial pressure values. Such instability in both systolic and detrusor pressures limit bladder compliance, as evidenced by repeated reflexive contractions resulting in incontinence. Note, participants' blood pressure was closely monitored during testing as well as signs and symptoms of autonomic dysreflexia. There were no complications as a result of the increased systolic blood pressure response during urodynamics. Following BC-scES mapping, stable detrusor filling pressure (<10 cmH₂O) with increased volume representative of improved bladder compliance and increased sphincter EMG activation for maintenance of urinary continence was achieved (FIG. 15, Panel B). Systolic blood pressure also remained stable (i.e. 110-120 mmHg) with optimal BC-scES.

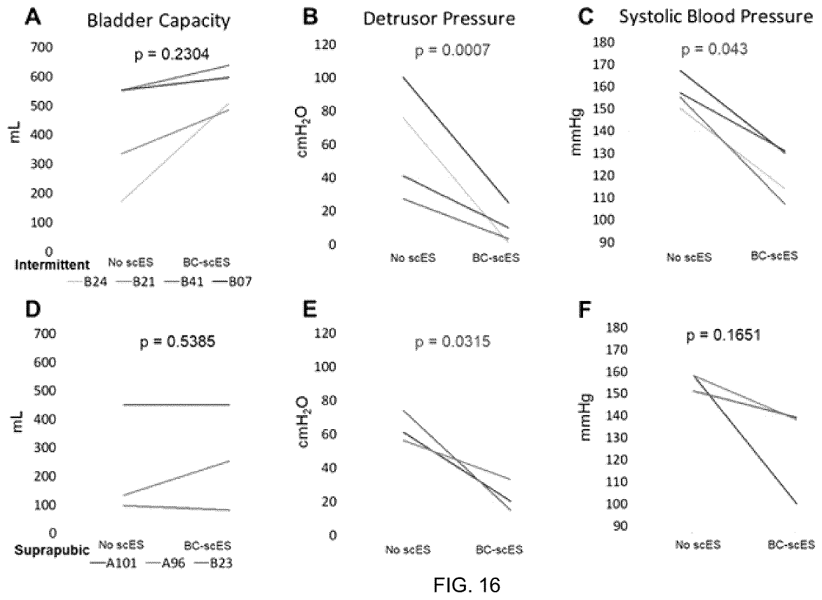

Referring now to FIG. 16, optimal BC-scES parameters that improved bladder capacity, while reducing maximum detrusor pressure and systolic blood pressure responses to bladder distention were compared across participants relative to outcomes obtained without scES (FIG. 16, Panels A-F). Post-mapping optimal BC-scES parameters in the CIC group resulted in significant improvements (reduction) in maximum detrusor pressure (p=0.0007) and maximum systolic blood pressure values (p=0.043) relative to no scES (FIG. 16, Panels B and C). Post-mapping optimal BC-scES parameters in the SP group resulted in a significant reduction in detrusor pressure relative to no scES (p=0.0315) (FIG. 16, Panel E). Refer to Table 7 for the percent change in each outcome for both groups. Note that the effective BC-scES parameters were not always from the final mapping session. In all participants, improvements in detrusor pressure were achieved with high-frequency configurations (i.e. >60 Hz).

TABLE 7
Comparison of bladder mapping group outcomes without scES and
with targeted BC-scES.

| | | CIC | | | | SP | | | |
|---|---|---|---|---|---|---|---|---|---|
| Cystometry measurements | | | | % | | | | % | |
| at maximum capacity | | No scES | BC-scES | Change | p-value | No scES | BC-scES | Change | p-value |
| Bladder Capacity | Avg. + S.D. | 401 ± 184 | 563 ± 79 | 66.4 ± 88.5 | 0.2304 | 226 ± 195 | 308 ± 221 | 24.2 ± 57.1 | 0.5385 |
| (mL) | Range | 171-450 | 483-637 | | | 96-450 | 120-552 | | |
| Detrusor Pressure | Avg. ± S.D. | 61 ± 33 | 14 ± 20 | −84.5 ± 11.4 | 0.0007* | 64 ± 9 | 28 ± 5 | −62.7 ± 19.7 | 0.0315* |
| (cmH₂O) | Range | 27-100 | 1-43 | | | 56-74 | 23-33 | | |
| Systolic Blood | Avg. ± S.D. | 157 ± 7 | 121 ± 13 | −23.4 ± 5.9 | 0.0043* | 156 ± 4 | 139 ± 15 | −19.1 ± 15.4 | 0.1651 |
| Pressure (mmHg) | | | | | | | | | |

(*, indicates significance);
Avg., Average; BC, bladder compliance;
CIC, clean intermittent catheterization;
cmH₂O, centimeters of water; ml, milliliters;
mmHg, millimeters of mercury;
scES, spinal cord epidural stimulation;
S.D., standard deviation;
SP, suprapubic catheter. Bladder outcomes, all normally distributed per Kolmogorov-Simonov test, were evaluated using paired t-test. All tests were 2-sided with a significance set to 5%.

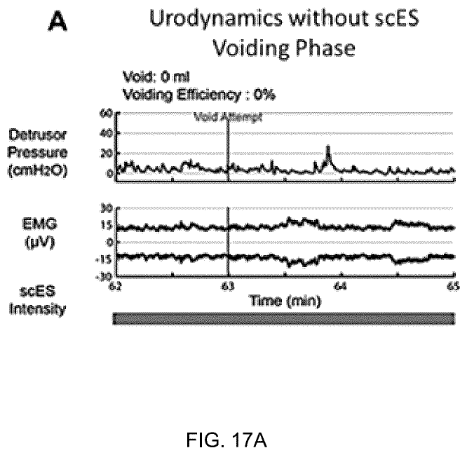

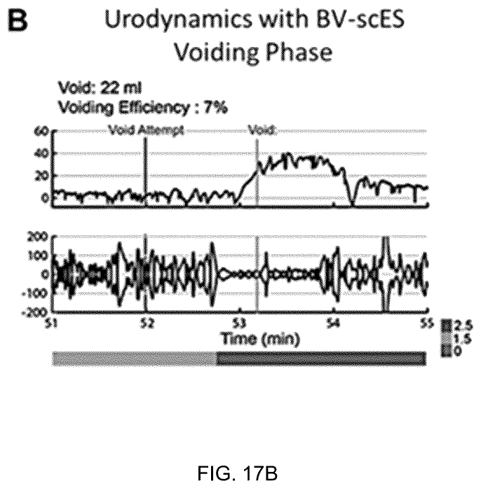

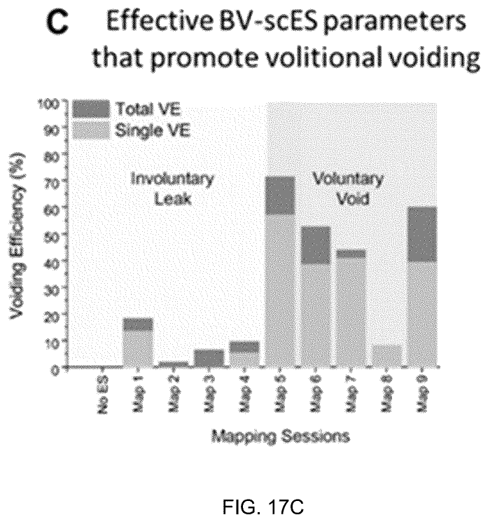

Referring now to FIGS. 17A-C, subsequent mapping for bladder void initiation (BV-scES) was evaluated during filling cystometry at 80% of filling capacity. Voiding was not achieved without scES in any of the participants. An example cystometry recording of the void attempt without scES is shown in FIG. 17A. The initiation of voiding with scES was achieved in participants when timed to intent and the desire to void with the sensation (direct or indirect) of bladder fullness (example, FIG. 17B), demonstrating the generation of a detrusor contraction and concurrent relaxation of the sphincter during voiding. Importantly, the void is timed close to the initiation of attempt generating a detrusor contraction from a low-pressure baseline and subsequent return to baseline after voiding. Effective BV-scES parameters were sufficient to generate the initiation of voiding with varying degrees of voiding efficiency (FIG. 17C). Voiding efficiency differences were a result of involuntary reflexive bladder contractions (FIG. 17C, Maps 1-4) relative to when the initiation of voiding was timed to voluntary intent (FIG. 17C, Maps 5-9). BV-scES mapping identified configurations that were frequency-dependent, and distinct from BC-scES, with the initiation of voiding occurring at low frequencies, between 25-30 Hz, for 6/7 participants.

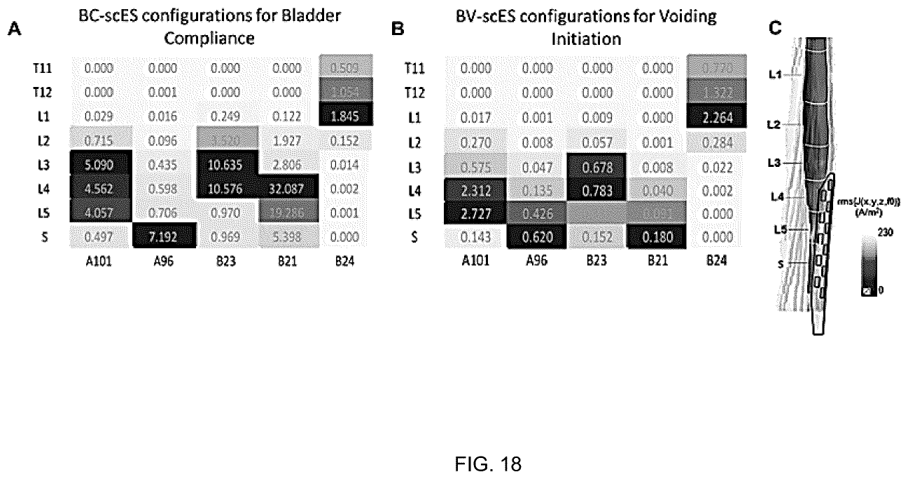

The maximum amount of electric charge per second delivered to the spinal cord and corresponding levels targeted by effective BC-scES and BV-scES parameters (for participants with available high-resolution MRI data) are illustrated in FIG. 18, Panels A and B, respectively. Activation of the rostral (spinal cord level L1) to mid-lumbosacral enlargement (spinal cord levels L3-L4) overlapped for ⅘ participants using effective BC-scES, while activation of the caudal (sacral) region of the lumbosacral enlargement was effective for bladder compliance for 1 participant (FIG. 18, Panel A). Activation of the caudal region of lumbosacral enlargement (spinal cord levels L4-S1) using BV-scES overlapped for ⅘ participants (FIG. 12B). Less charge was required for a void effect relative to a storage effect. An example of an MRI-based 3D model of the spinal cord at the lumbosacral enlargement for participant B21 and the location of the scES paddle array with respect to the spinal cord is shown in FIG. 18, Panel C, where the distribution of the current density targets the caudal region of the lumbosacral enlargement.

The current study investigated the effects of scES on bladder function through functional mapping during filling cystometry. Placement of electrodes and the direction/extent of current spread on the spinal cord targeted by scES bladder cohorts was also performed using high-resolution MRI and computational modeling to better understand the neuroanatomical regions responsible for mediating improvements in LUT function. For those individuals with available high-resolution MRI (n=5), neuromodulation of bladder compliance was primarily effective when scES targeted spinal cord regions L3-L4, while initiation of voiding was enhanced at caudal regions of the lumbosacral spinal cord (L4-S1). Storage and void effects were frequency-dependent, with high-frequency cohorts mediating bladder compliance and low-frequency cohorts mediating void initiation. Intensity selection for storage and voiding was subject-specific.

Epidural stimulation targeting the lumbosacral spinal cord has been shown in multiple studies to improve bladder function in humans with chronic SCI, even when stimulation was not directly optimized for bladder function. Our previous work demonstrated that LUT function benefits from combined activity-based recovery training with scES targeted for either stepping/standing and cardiovascular/voluntary movement. While scES was not directly configured for bladder function, nor was stimulation “on” during cystometry, optimizing the state of excitability of the human spinal circuitry with scES and through the integration of appropriate sensory information with task-specific training, may have led to improved adaptations in detrusor activity and reciprocal somatic facilitation of the sphincter. However, blood pressure was not entirely stabilized in response to bladder distention. Here we show that scES targeting bladder compliance also maintained systolic blood pressure within normative ranges during cystometry, likely due to suppression of detrusor overactivity at larger storage volumes.

Cardiovascular disturbances after SCI, such as autonomic dysreflexia have been reported to occur up to 40 times a day in susceptible individuals (primarily SCI above T6), with neurogenic detrusor overactivity being one of the primary triggers. We and others have shown that cardiovascular instability in response to bladder filling is widespread after SCI, also occurring in those with injuries below T6, as the sympathetic outflow extends to L2. The ability to recover bladder function such as, increasing bladder capacity, minimizing detrusor instability, and improving bladder pressure and emptying, is limited by such severe fluctuations in blood pressure. Regulating blood pressure in those having SCI is challenging and is exacerbated by current methods of monitoring bladder function, which is limited to testing only in a clinical urodynamics laboratory. The extreme dysregulation of cardiovascular and bladder function underscores the importance of addressing these multi-faceted autonomic complications.

Assessment of urodynamic parameters during BC-scES mapping revealed improvements in detrusor filling pressure and maintenance of tonic sphincter EMG activity, which resulted in improved bladder compliance and stabilization of systolic blood pressure that was most effective in the upper to mid lumbar regions, with L3-L4 segments appearing to be a key region. The activity of the detrusor muscle and external urethral sphincter (EUS) can be coordinated and modulated by neural circuits located in the spinal cord as well as from supraspinal centers. Pre-clinical research in rodents suggests evidence of a lumbar coordinating center at L3-L4 spinal segments that contributes to the emergence of EUS bursting and detrusor-sphincter coordination after SCI. Similarly, neurons involved in mediating the ejaculation reflex, which involves periurethral muscles important for sphincter coordination and anterograde semen propulsion, have been identified in the L3-4 spinal segments in rats and from L3-L5 in humans. Furthermore, activation of the dorsal surface of the spinal cord with stimulation at L3 in rodents has been shown to reduce detrusor overactivity and effectively modulate EUS activity, reducing urethral resistance and promoting emptying.

Activation of the rostral array targeting upper lumbar sympathetic (L1-L2) regions of the spinal cord may also help facilitate urinary storage, promoting low intravesical pressures and relaxing the detrusor muscle during bladder filling. As the bladder accommodates a larger volume, afferent fiber activation initiates an intersegmental spinal reflex pathway from the sacral cord to thoracolumbar sympathetic neuron, which stimulates contraction of the internal urethral sphincter and inhibits bladder activity. The storage effect is enhanced, as intrinsic viscoelasticity of the detrusor muscle permits the bladder wall to accommodate increasing volume, while the parasympathetic pathway remains quiescent.

In one participant (A96), a positive storage effect was achieved by stimulation at conus. Somato-visceral sacral reflexes that remain intact after suprasacral SCI can be effectively targeted to neuromodulate bladder function. Stimulation-induced activation of pudendal afferents, which project onto sympathetic and parasympathetic pathways can lead to inhibition of the detrusor muscle, suppression of overactivity, and simultaneous excitation of the sphincters, resulting in a coordinated storage response. Indeed, numerous studies in humans with SCI have shown that stimulation of branches of the pudendal nerve suppresses bladder hyperreflexia, reducing incidences of incontinence and improving bladder capacity. Similar to mechanisms involved in pain control with spinal cord stimulation, antidromic facilitation of somatic fibers to promote a sphincter guarding response, as well as orthodromic activation of ascending sensory dorsal column fibers to upper and mid-lumbar segments, including interneuronal segmental connections, may contribute to the storage effect with scES at distal segments.

The results of the present study also expand the scope of our earlier investigations in which we identified scES parameters that improved the efficiency of the reflexive void after motor complete SCI. Similarly, we identified low-frequency BV-scES parameters that were used to augment the volitional intent to void and were driven by sensations of bladder fullness at 80% capacity. Each participant was able to initiate voluntary voiding in the presence of scES. Lumbosacral BV-scES may be used to enable the spinal cord, below the level of injury, to efficiently integrate afferent sensory information from the bladder, along with residual signals from the pontine micturition center, to generate inhibitory input to sympathetic and somatic regions of the spinal cord. The initiation of voiding was most effective at caudal segments of the spinal cord where local spinal reflexes involved in sphincter coordination could be modulated to decompress the sphincter during intent and with sufficient bladder volume, simultaneously facilitate parasympathetic activation of the detrusor muscle. During void attempts, an increase in intra-abdominal pressure (indirectly measured via the rectal catheter during urodynamics) may assist with decompression of urethral resistance at the bladder neck and proximal urethra to initiate emptying. It's important to note that the initiation of voiding began from low detrusor pressure and while detrusor pressure rose during void attempts and was sufficient to override the pressure generated from urethral sphincters, it did not remain sustained, which could result in vesicoureteral reflux. While the focus of this study was to initiate voiding, future work is aimed at implementing BV-scES training to improve the efficiency of the void. Even though inhibition of EUS activity during micturition is partly dependent on supraspinal mechanisms, once the initiation of urine flows through the urethra, voiding is facilitated by a urethral-to-bladder reflex and increased efferent excitatory outflow to the bladder via pelvic nerves. BV-scES training can be used to strengthen these local circuits.

Understanding the parameters of stimulation and programming strategies represents an important and necessary step in advancing neuromodulation targeted at improving autonomic function after SCI. Neuroimaging and image-based computational modeling of the spinal cord, nerve roots, and surrounding tissue can optimize outcomes for stimulation-based interventions, such as scES. This disclosure has shown that maximizing the coverage of the excitable tissues by electrical stimulation at relevant levels of the spinal cord can improve functional outcomes. Also, the length of the spinal cord and location of spinal cord segments with respect to the vertebrae, particularly at lumbosacral enlargement, varies across individuals with the location of the conus tip ranging between T12 to L2. Therefore, relying only on the vertebral levels as a guide for stimulating specific spinal cord regions could often be inadequate and not lead to the best possible outcomes, limiting the understanding of the mechanism of action of electrical stimulation as a neuromodulation intervention. The differences in spinal cord level of activation across participants may also be due to the level at which the electrode array was placed and available tissue accessible for scES.

The results of the current study demonstrate that scES can be used to simultaneously and safely control urinary continence and the initiation of voiding while managing distention-associated dysregulation of blood pressure. Importantly, these initial findings reveal the complex dynamics and interplay between sympathetic and parasympathetic circuitries that are being integrated and regulated within the spinal cord below the level of SCI. This spinal circuitry is driven by afferent input and modulated by scES to effectively optimize the state of the bladder and associated systemic blood pressure responses. It is also likely, given the void intent results, that scES enhances the conduction properties of residual damaged or non-functional but anatomically intact long ascending/descending axons that are traversing across the spinal injured segment. In this manner, scES acting upon lumbosacral spinal neural networks can promote an increase in overall autonomic regulation sufficient to interact with appropriate sensory cues (e.g. from bladder distention) as well as engage descending supraspinal residual inputs (e.g. intent to void) to facilitate continued involvement of such networks to maintain target bladder compliance, initiate on-demand voiding, and regulate cardiovascular parameters during storage and emptying. Current studies in progress are evaluating the integration of BC-scES and BV-scES in the home setting in order to understand the natural transition from storage to voiding.

Seven individuals (32.1±4.6 years of age; 6:1, male: female) with motor complete SCI (C3-T2) participated in a research study conducted at the University of Louisville investigating the effects of scES directly targeted to improve bladder storage and emptying between the years of 2018-2021. Participants were already surgically implanted with a 16-electrode array (5-6-5 Specify, Medtronic, Minneapolis, MN, USA) at the T11-L1 vertebral levels over spinal cord segments L1-S1. The electrode lead was tunneled subcutaneously and connected to the pulse generator (RestoreADVANCED (B21, B23), or Intellis (A101, A96, A68, B24, B07), Medtronic, Minneapolis, MN) placed ventrally in the abdomen. All research participants were over 21 years of age at the time of scES implant and met the following inclusion criteria: non-progressive SCI at the cervical and upper thoracic spinal cord, AIS A or B, and at least two years post-injury with no medical conditions unrelated to SCI at the time of implant.

Participants received a clinical evaluation prior to study participation to assess motor and sensory status. Two clinicians independently performed the International Standards for Neurological Classification of Spinal Cord Injury in order to classify participants' injuries using the ASIA (American Spinal Injury Association) Impairment Scale (AIS). A physical examination and bladder/kidney ultrasound were performed by the study physician and study urologist, respectively, for medical clearance, ensuring participation safety using the following inclusion criteria: 1) stable medical condition; 2) no painful musculoskeletal dysfunction, unhealed fracture, contracture, pressure sore or urinary tract infection that might interfere with training; 3) no untreated psychiatric disorders or ongoing drug abuse; 4) clear indications that the period of spinal shock is concluded determined by the presence of muscle tone, deep tendon reflexes or muscle spasms and discharged from standard inpatient rehabilitation; 5) non-progressive supra-sacral SCI; 6) bladder dysfunction as a result of SCI; and 7) epidural stimulator implanted at the lumbosacral spinal cord. None of the participants had ever received Botox injections for management of bladder dysfunction and all participants were off anti-spasticity medication (e.g. Baclofen). None of the participants altered their method of bladder emptying throughout the study.

All data were obtained from standard urodynamic evaluations with recommendations from the International Continence Society. Using the Aquarius® LT system (Laborie, Williston, VT), cystometry was performed in the seated position via a single sensor, dual-channel catheter (7 Fr, T-DOC® Air-Charged™, Laborie, Williston, VT) with the continuous filling of sterile, body-temperature saline (37° C.) at a fixed rate of 10 mL/min, more closely reflecting physiological filling. Abdominal pressure was measured via a rectal catheter (7 Fr, T-DOC® Air-Charged™, Laborie, Williston, VT). Pelvic floor EMG (Neotrode II, Laborie, Williston, VT) was recorded using surface patch EMG electrodes and a grounding pad was placed on a bony prominence, usually the hip or knee. Note that to distinguish between isolated EMG activation of the intramuscular urethral striated sphincter versus general muscle activation of the pelvic floor, an intramural needle electrode EMG is necessitated. However, given the ethical concerns of repeated needle electrode placement, surface electrodes are consistently used in daily clinical practice as an established method for diagnosis of lower urinary tract dysfunction. Detrusor pressures were calculated by subtracting the intra-abdominal pressure from the intra-vesical pressure. Research participants were asked to cough to verify catheter positions. Prior to the start of filling, scES amplitude was ramped up slightly to isolate the initial targeted location (pelvic floor, bladder, abdominal region versus legs, feet—see Mapping section for details below). During the filling phase of the experiment, participants were instructed to communicate bladder sensations (first sensation); the desire to urinate (first urge to void); and strong desire to void and the feeling that voiding/leaking cannot be delayed (maximum capacity). Given that many SCI participants may have a loss of bladder sensation, indirect sensations were also used. The volume of water infused and bladder pressure were continuously recorded. Uninhibited bladder contractions also were identified. Blood pressure (BP) and heart rate (HR) were obtained from the brachial artery, measured by oscillometric technique (Carescape V100, GE Healthcare, Milwaukee, WI), throughout the urodynamic session. Baseline BP recordings were obtained in the supine and seated positions prior to urodynamic testing. Any signs and self-reported symptoms of autonomic dysreflexia were documented and observed throughout testing. Bladder filling was ceased and the bladder was emptied if any of the following conditions were observed: 1) spontaneous urine leakage, 2) filling 600 ml or reaching maximum bladder capacity as evidenced by a rise in the compliance curve, 3) high sustained intravesical pressure ≥40 cmH2O or, 4) autonomic dysreflexia as evidenced by a sustained systolic blood pressure recording of 20 mm Hg from baseline and/or intolerable symptoms. A post-fill BP recording was captured to ensure BP values returned to baseline.

During the voiding phase, a “permission to void” command followed after stopping the infusion pump (at approximately 80% capacity). Detrusor pressure was monitored during the void attempt and uroflowmetry for the voided volume. Post-void residual volume was measured to evaluate the extent of bladder emptying. Importantly, natural diuresis occurs during cystometry and may contribute to measured bladder volumes. Note, that voiding not was attempted if blood pressure and detrusor pressure were elevated, as indicated above.

Bladder capacity was calculated as the volume of leaked or voided fluid plus any residual amount removed from the bladder. Voiding efficiency (VE) was calculated as: VE=[volume voided/(volume voided+residual volume)×100]. Compliance was calculated by dividing the volume change (ΔV) by the change in detrusor pressure (ΔPdet) during that change in bladder volume and was expressed in ml/cm H2O. The intravesical pressure (Pves) at which involuntary expulsion of water/urine from the urethral meatus was observed was considered the detrusor leak point pressure (DLPP). Maximum detrusor pressure (MDP) was identified as the peak detrusor pressure during the voiding phase of the cystometrogram. Detrusor pressures were calculated by subtracting the intra-abdominal pressure from the intra-vesical pressure. Note, if a participant did not leak during the filling cycle, MDP was used in place of DLLP. All analyses were performed with customized software in MATLAB (MathWorks, Natick, MA).

scES was administered by a multi-electrode array implanted in the epidural space over the dorsum of the spinal cord. An implanted package containing stimulating circuits, a rechargeable battery, and wireless communication activates the electrodes (16 platinum electrodes arranged in three columns of [5-6-5], Medtronic Inc.). The pattern of electrically active electrodes, as well as electrode voltage, stimulating frequency, and stimulating pulse width was remotely programmed. Since different spatial activation patterns and different frequency parameters affect different spinal circuits, the electrode array was reconfigured, within limits, to bias its facilitating effects toward bladder storage and emptying. Bladder mapping followed a human-guided interactive optimization approach where the experimental mapping process was subdivided into separate domains/tasks to isolate parameters for storage function and the initiation of voiding. Since these domains are inter-dependent, subsequent optimization tested and refined parameters concurrently in order to build comprehensive cohorts for multi-system stimulation. Each participant completed a minimum of 20 urodynamic sessions (10 for storage; 10 for void initiation) mapping the detrusor and urethral pressure responses as well as sphincter EMG responses during both filling and emptying cystometry phases while scES parameters (anode, cathode selection; frequency and amplitude, and the number of cohorts) were modulated to isolate successful configurations. The goal for bladder capacity (BC)-scES was to target volumes between 400-500 mL based on average normal capacity and avoiding over-distention in individuals performing intermittent catheterization 4-6 times/day (including average fluid intake). Also targeted were filling pressures (<10 cmH₂O) to improve overall bladder compliance and detrusor leak-point pressures (<40 cmH₂O). Maintaining normative systolic pressures during filling, within a range of 110-120 mmHg, was a further goal. All enrolled participants completed prior scES mapping studies for cardiovascular function and thus, the cardiovascular cohort was integrated if blood pressure was elevated. All simulations accounted for any cardiovascular cohorts.

Based on previously published methods, bladder mapping was performed by selecting electrode configurations with cathodes positioned caudally, targeting the sacral micturition center and parasympathetic pathways, then with cathodes positioned in the mid-array to target the purported lumbar spinal coordinating center with presynaptic connections to sphincter motoneurons, and then with cathodes positioned rostrally, to target sympathetic pathways (location selection order varied for each mapping session). Changes in detrusor pressure, sphincter activation/relaxation, and blood pressure responses were monitored during bladder filling while conducting a gradual ramp-up of stimulation frequency and intensity until a near-motor threshold stimulation amplitude that did not elicit direct lower limb movements was selected. Stimulation frequency and intensity were then modulated synergistically in order to isolate an optimal frequency that elicited an overall continuous low detrusor pressure filling profile with a synchronized sphincter EMG pattern effective for bladder continence. Guided by participants' sensations of bladder fullness, the transition from continence to micturition aimed to integrate ascending inputs and descending volitional drive. Electrodes in the caudal, middle, and rostral regions of the array were selected while the frequency was kept fixed and amplitude adjusted in order to isolate an optimal intensity that drove the initiation of voiding activity (simultaneous increase in detrusor pressure with a decrease in upper urethral pressure and quiescence of sphincter EMG responses). Electrode location and selection refinement was further modified to adjust for sensory and autonomic symptoms during mapping.

Lower extremity and trunk EMG was monitored continuously throughout mapping to identify those parameters that modulate detrusor pressure and coordination with the external anal sphincter muscle (mirroring external urethral sphincter) and blood pressure but do not elicit motor activity in the lower extremity or trunk. Stimulation amplitude was lowered and electrode selection was modified to inhibit lower extremity/trunk activity. EMG was collected at 2,000 Hz using a 24-channel hard-wired AD board and custom-written acquisition software (Labview, National Instruments, Austin, TX, United States). EMG (MotionLab Systems, Baton Rouge, LA, United States) from the soleus, medial gastrocnemius, tibialis anterior, medial hamstrings, rectus femoris, and vastus lateralis using bipolar surface electrodes with fixed inter-electrode distance. In addition, two surface electrodes were placed over the paraspinal muscles, symmetrically lateral to the epidural electrode array incision site. These two electrodes were used to record the stimulation artifact from the implanted electrode. EMG data were rectified and high-pass filtered at 40 Hz using Labview software customized by our laboratory. All mapping urodynamic sessions were conducted at least two days apart.

MRI 2-D scans of all levels of the spine with high spatial resolution were recorded using either Siemens 3.0 Tesla Magnetom Skyra or Siemens 1.5 Tesla ESPREE in sagittal and axial planes. Sagittal images were obtained in two or three separate sequences (depending on the height of the participant) to cover the whole spine from the foramen magnum to the end of the sacral region. These images were reviewed by the radiologist and neurosurgeon to screen for syrinxes, significant stenosis, scoliosis, level of injury and stabilizing treatment, and related surgical changes over time.

Axial images were obtained using T2 Turbo Spin Echo in 4 to 5 separate sequences (depending on the height of the participant) with a focused field of view typically from cervical, upper thoracic, mid thoracic, lower thoracic-upper lumbar, and lower lumbosacral levels. Axial images were obtained with 3 mm slice thickness and zero mm gap. The axial images were used to measure the cross-sectional area of the spinal cord at different vertebral levels and to reconstruct a 3-D individual-specific model of the lumbosacral enlargement necessary for anatomical mapping of the L1-S1 spinal cord segments (described below).

The anterior-posterior and lateral X-ray images of the spinal cord at the location of the scES paddle electrode implant, obtained after implantation from each participant, were used to identify the T12 vertebra based on the location of the last floating rib, and identify the exact location of the rostral and caudal ends of the paddle electrode with respect to the vertebral body. The location of the paddle was estimated with respect to the spinal cord by integrating the lateral X-ray with the sagittal and axial MRI scans. Based on the length of the paddle electrode (46.5 mm for Medtronic Specify® 5-6-5 lead), 15 MRI axial slices (total of 15×3 mm=45 mm in length) that best describe this location were identified. The paddle electrode was placed on the 3-D model based on the location of the identified 15 axial slices.

The 3-D reconstructed model was completed for 5 out of the 7 participants who had high-resolution MRI scans with incorporation of the finite element modeling technique and neuronal activation function to investigate the distribution patterns of the electric fields generated by scES. Recording MRI axial scans with high spatial resolution enables one to locate and trace the dorsal and ventral nerve roots that float in the cerebrospinal fluid. As the spinal cord typically ends at the L1 vertebral level, the nerve roots that enter the spinal cord at the lumbosacral enlargement elongate to exit the spinal canal further distally at the corresponding vertebral levels (L1-S1). The spinal cord lumbar segments assigned to L1-S1 were anatomically estimated by identifying the set of nerve roots that exit the spinal canal at each vertebral level and back-tracing those nerve roots into the spinal cord body. Furthermore, the axial images of the lumbosacral spinal cord were segmented based on the area of the cerebrospinal canal, spinal cord tissue, and nerve roots. A 3-D model of the spinal cord of each individual was then reconstructed using custom-written codes in MATLAB. The estimated neuroanatomical levels of the spinal cord were visualized on the 3-D reconstructed model of the lumbosacral region.

A set of computational tools were used to map the human spinal cord for bladder function. This toolset included modules from Sim4Life, MANGO, and custom-written programs in MATLAB and Python. Finite element analysis included model creation and generation of the topology and geometry information representing the spinal cord and surrounding tissue boundaries using Sim4Life. The meshing phase decomposed the model geometry into simple shapes or voxels that fill the volume. Each voxel has its own electric field conductivity parameters and initial conditions. Partial differential equations specified electric field distribution among voxels based on the material properties. Features and trends generated from the solutions output from simulations were summarized for each participant. Post-processing produced data products from the instantaneous electric field solution of each stimulation pulse, including visualizations of the fields and current density superimposed on the geometry and along a line traversing the dorsomedial surface of the spinal cord. These results were used to calculate quantiles such as maximums, minimums, averages, and integrals over points. The amount of electric charge per second (in Coulomb per second per square meter) delivered to each level of the lumbosacral spinal cord was calculated by multiplying the amount of current density (ampere per square meter), as determined by the finite element modeling, with the stimulation frequency (hertz) and pulse width (seconds). Data and graphics were exported for illustration and used by MATLAB- and Python-based programs.

Bladder outcomes, all normally distributed per Kolmogorov-Simonov test, were evaluated using paired t-test. All tests were 2-sided with a significance set to 5%. Statistical analyses were performed in SAS 9.4 (SAS Inc., Cary, NC).

Various aspects of different embodiments of the present disclosure are expressed in paragraphs X1 and X2 as follows:

X1. One embodiment of the present disclosure includes a method for configuring neurostimulation, comprising selecting one or more desired physiological functions; creating a 3D model of at least a portion of a spinal cord, the modeled portion of the spinal cord including a lumbosacral enlargement; calculating, based at least in part on the 3D model, for at least one position of an electrode array including a plurality of electrodes, a portion of the lumbosacral enlargement covered by the electrode array at the at least one position; positioning the electrode array with respect to the spinal cord, the positioning based in part on the calculated portion of the lumbosacral enlargement covered by the electrode array at the at least one position and based in part on the selected one or more desired physiological functions; evoking potentials by applying spinal cord stimulation using the electrode array and determining stimulation thresholds to evoke responses from said spinal cord stimulation; mapping a spatial relationship between the plurality of electrodes and the evoked responses; and selecting specific electrodes within the plurality of electrodes for activation based on activation of said specific electrodes evoking responses promoting the one or more desired physiological functions.

X2. Another embodiment of the present disclosure includes a method for applying neurostimulation to an individual, comprising selecting one or more desired physiological functions; creating a 3D model of at least a portion of a spinal cord, the modeled portion of the spinal cord including a lumbosacral enlargement; calculating, based at least in part on the 3D model, for at least one position of an electrode array including a plurality of electrodes, a portion of the lumbosacral enlargement covered by the electrode array at the at least one position; positioning the electrode array with respect to the spinal cord, the positioning based in part on the calculated portion of the lumbosacral enlargement covered by the electrode array at the at least one position and based in part on the selected one or more desired physiological functions; evoking potentials by applying spinal cord stimulation using the electrode array and determining stimulation thresholds to evoke responses from said spinal cord stimulation; mapping a spatial relationship between the plurality of electrodes and the evoked responses; selecting specific electrodes within the plurality of electrodes for activation based on activation of said specific electrodes evoking responses promoting the one or more desired physiological functions; and applying spinal cord stimulation using the specific electrodes to promote the one or more desired physiological functions.

Yet other embodiments include the features described in any of the previous paragraphs X1 or X2, as combined with one or more of the following aspects:

Wherein the evoked responses are motor muscle responses, non-motor muscle responses, or non-muscle responses.

Wherein the method includes applying spinal cord stimulation below the determined stimulation thresholds using the specific electrodes to promote the one or more desired physiological functions.

Wherein the evoked responses are motor muscle responses.

Wherein the method includes applying spinal cord stimulation at or above the determined stimulation thresholds using the specific electrodes to promote the one or more desired physiological functions.

Wherein the evoked responses are non-motor muscle responses or non-muscle responses.

Wherein the lumbosacral enlargement includes a plurality of lumbosacral spinal cord levels; wherein each evoked response is evoked by applying spinal cord stimulation to one or more levels of the plurality of spinal cord levels; and wherein positioning the electrode array includes positioning the electrode array such that the calculated portion of the lumbosacral enlargement covered by the electrode array includes each spinal cord level stimulated in evoking each evoked response promoting the one or more desired physiological functions.

Wherein the electrode array is positioned at a transcutaneous position.

Wherein the electrode array is positioned at an epidural position.

Wherein the method includes applying spinal cord stimulation using the specific electrodes to promote the one or more desired physiological functions.

Wherein the method includes repositioning the electrode array based on said evoking potentials.

Wherein said repositioning occurs after said evoking potentials and before said mapping.

Wherein the one or more desired physiological functions is a plurality of non-identical physiological functions.

Wherein the method includes applying spinal cord stimulation using the specific electrodes to promote one of the plurality of desired physiological functions then subsequently applying spinal cord stimulation using the specific electrodes to promote a different one of the plurality of desired physiological functions

Wherein the one or more desired physiological functions is one or more of standing, sitting, walking, increasing blood pressure, decreasing blood pressure, maintaining blood pressure, voiding bladder, refraining from voiding bladder, voiding bowels, refraining from voiding bowels.

Wherein the one or more desired physiological functions is a plurality of standing, sitting, walking, increasing blood pressure, decreasing blood pressure, maintaining blood pressure, voiding bladder, refraining from voiding bladder, voiding bowels, refraining from voiding bowels.

Wherein mapping the spatial relationship between the plurality of electrodes and the evoked responses comprises activating combinations of electrodes within the plurality of electrodes and determining the response evoked from such activation.

Wherein the method further includes, after said mapping the spatial relationship between the plurality of electrodes and the evoked responses, mapping a relationship between the plurality of electrodes and the one or more desired physiological functions.

Wherein mapping the relationship between the plurality of electrodes and the one or more desired physiological functions comprises activating combinations of electrodes within the plurality of electrodes evoking responses promoting the one or more desired physiological functions and determining if the one or more desired physiological functions is achieved.

The foregoing detailed description is given primarily for clearness of understanding and no unnecessary limitations are to be understood therefrom for modifications can be made by those skilled in the art upon reading this disclosure and may be made without departing from the spirit of the invention.

1. A method for configuring neurostimulation, comprising:
selecting one or more desired physiological functions;
creating a 3D model of at least a portion of a spinal cord, the modeled portion of the spinal cord including a lumbosacral enlargement;
calculating, based at least in part on the 3D model, for at least one position of an electrode array including a plurality of electrodes, a portion of the lumbosacral enlargement covered by the electrode array at the at least one position;
positioning the electrode array with respect to the spinal cord, the positioning based in part on the calculated portion of the lumbosacral enlargement covered by the electrode array at the at least one position and based in part on the selected one or more desired physiological functions;
evoking potentials by applying spinal cord stimulation using the electrode array and determining stimulation thresholds to evoke responses from said spinal cord stimulation;
mapping a spatial relationship between the plurality of electrodes and the evoked responses; and
selecting specific electrodes within the plurality of electrodes for activation based on activation of said specific electrodes evoking responses promoting the one or more desired physiological functions.

2. The method of claim 1, wherein the evoked responses are motor muscle responses, non-motor muscle responses, or non-muscle responses.

3. The method of claim 2, further comprising applying spinal cord stimulation below the determined stimulation thresholds using the specific electrodes to promote the one or more desired physiological functions.

4. The method of claim 3, wherein the evoked responses are motor muscle responses.

5. The method of claim 2, further comprising applying spinal cord stimulation at or above the determined stimulation thresholds using the specific electrodes to promote the one or more desired physiological functions.

6. The method of claim 5, wherein the evoked responses are non-motor muscle responses or non-muscle responses.

7. The method of claim 1,
wherein the lumbosacral enlargement includes a plurality of lumbosacral spinal cord levels;
wherein each evoked response is evoked by applying spinal cord stimulation to one or more levels of the plurality of spinal cord levels; and
wherein positioning the electrode array includes positioning the electrode array such that the calculated portion of the lumbosacral enlargement covered by the electrode array includes each spinal cord level stimulated in evoking each evoked response promoting the one or more desired physiological functions.

8. The method of claim 1, wherein the electrode array is positioned at a transcutaneous position.

9. The method of claim 1, wherein the electrode array is positioned at an epidural position.

10. The method of claim 1, further comprising applying spinal cord stimulation using the specific electrodes to promote the one or more desired physiological functions.

11. The method of claim 1, further comprising repositioning the electrode array based on said evoking potentials.

12. The method of claim 1, wherein said repositioning occurs after said evoking potentials and before said mapping.

13. The method of claim 1, wherein the one or more desired physiological functions is a plurality of non-identical physiological functions.

14. The method of claim 13, further comprising applying spinal cord stimulation using the specific electrodes to promote one of the plurality of desired physiological functions then subsequently applying spinal cord stimulation using the specific electrodes to promote a different one of the plurality of desired physiological functions.

15. The method of claim 1, wherein the one or more desired physiological functions is one or more of standing, sitting, walking, increasing blood pressure, decreasing blood pressure, maintaining blood pressure, voiding bladder, refraining from voiding bladder, voiding bowels, refraining from voiding bowels.

16. The method of claim 15, wherein the one or more desired physiological functions is a plurality of standing, sitting, walking, increasing blood pressure, decreasing blood pressure, maintaining blood pressure, voiding bladder, refraining from voiding bladder, voiding bowels, refraining from voiding bowels.

17. The method of claim 1, wherein mapping the spatial relationship between the plurality of electrodes and the evoked responses comprises activating combinations of electrodes within the plurality of electrodes and determining the response evoked from such activation.

18. The method of claim 1, further comprising, after said mapping the spatial relationship between the plurality of electrodes and the evoked responses, mapping a relationship between the plurality of electrodes and the one or more desired physiological functions.

19. The method of claim 18, wherein mapping the relationship between the plurality of electrodes and the one or more desired physiological functions comprises activating combinations of electrodes within the plurality of electrodes evoking responses promoting the one or more desired physiological functions and determining if the one or more desired physiological functions is achieved.

20. A method for applying neurostimulation to a subject, comprising:
selecting one or more desired physiological functions;
creating a 3D model of at least a portion of a spinal cord, the modeled portion of the spinal cord including a lumbosacral enlargement;
calculating, based at least in part on the 3D model, for at least one position of an electrode array including a plurality of electrodes, a portion of the lumbosacral enlargement covered by the electrode array at the at least one position;
positioning the electrode array with respect to the spinal cord, the positioning based in part on the calculated portion of the lumbosacral enlargement covered by the electrode array at the at least one position and based in part on the selected one or more desired physiological functions;
evoking potentials by applying spinal cord stimulation using the electrode array and determining stimulation thresholds to evoke responses from said spinal cord stimulation;
mapping a spatial relationship between the plurality of electrodes and the evoked responses;
selecting specific electrodes within the plurality of electrodes for activation based on activation of said specific electrodes evoking responses promoting the one or more desired physiological functions; and
applying spinal cord stimulation using the specific electrodes to promote the one or more desired physiological functions.
